(19) United States
(12) Patent Application Publication
(10) Pub. No.: US 2023/0211159 A1
(43) Pub. Date: Jul. 6, 2023

(54) CUSTOMIZED TACS (TRANSCRANIAL ALTERNATING CURRENT STIMULATION) APPARATUS AND METHOD FOR STIMULATING A BRAIN WAVE BY ENTRAINING SYNCHRONIZED OSCILLATION BASED ON REAL-TIME EEG SIGNAL MONITORING

(71) Applicant: Lee Sol Co., Ltd., Seoul (KR)

(72) Inventors: Seung Woo LEE, Seoul (KR); Juyeong PARK, Seongnam-si, (KR); Gusung KWON, Seoul (KR); Chanyuk PARK, Seoul (KR)

(73) Assignee: Lee Sol Co., Ltd., Seoul (KR)

(21) Appl. No.: 17/868,367

(22) Filed: Jul. 19, 2022

(30) Foreign Application Priority Data
Dec. 31, 2021 (KR) 10-2021-0194215
Mar. 25, 2022 (KR) 10-2022-0037069

(51) Int. Cl. A61N 1/36

(52) U.S. Cl. CPC A61N 1/36025; A61N 1/36031

(57) ABSTRACT

An apparatus includes a stimulation part that stimulates a brain of an object; a sensing part that measures a response of the brain to a stimulation; and a control part that determines whether a first response entraining oscillation synchronized in a plurality of brain areas of the object is derived or not, based on a measured brain response. The stimulation part is an electrical stimulation part that transfers a transcranial alternating current stimulation (tACS) and the transcranial alternating current stimulation is a first combined stimulation in which a signal is repeatedly ON/ OFF according to a predetermined first frequency and an ON signal according to the first frequency is applied as a stimulation according to a predetermined second frequency.

### CROSS-REFERENCE TO RELATED APPLICATIONS

This application claims priority and benefit of Korean Patent Application No. 10-2021-0194215 filed on Dec. 31, 2021 and Korean Patent Application No. 10-2022-0037069 filed on Mar. 25, 2022 in the Korean Intellectual Property Office, the entire disclosures of which are hereby incorporated by reference herein in its entirety.

### BACKGROUND

### Field

The present disclosure relates to a customized tACS(transcranial Alternating Current Stimulation) apparatus and a method for stimulating a brain wave by entraining synchronized oscillation based on real-time EEG signal monitoring.

### Description of the Related Art

It is the widely-accepted theory that possibility of the occurrence of a disease such as Alzheimer’s disease rises due to hardening of a brain blood vessel resulting from the progression of aging and the decline in arterial beating that is helpful for the circulation of a brain lymphatic system.

Further, dementia including Alzheimer’s disease (AD) is a fatal disease to the brain, characterized by deterioration in the brain and a cognitive function (Canter). It is known that various factors relates to AD pathogenesis including amyloid-β deposition, hyperphosphorylated tau accumulation, microgliacyte- and astrocyte-mediated inflammation, and losses of neuron and synapse.

AD falls in the field of unmet medical needs currently having no radical therapy, and constitutes a medical challenge in modern society.

Firstly, the aging society results in tendency of increase in the social/economic burden.

According to the increase in aging population, the average annual medical expenses for AD recorded a 17.1% increase during 2011 to 2015. AD management cost, which was about KRW 13.2 trillion (0.9% of GDP) as of 2015, is expected to increase 8 times in 2050, reaching KRW 106.5 trillion (3.8% of GDP).

World-wide social/economic expenses for AD was USD 818 billion (Approx. KRW 975.6 trillion, 1.09% of global GDP) as of 2015 and is expected to reach USD 2 trillion (Approx. KRW2,382 trillion) in 2030.

Next, currently there is a limitation to traditional medication for treating AD.

99.6% of drug candidates in clinical trials for AD fail. Global pharmaceutical companies lead new drug development at enormous expense but repeat fails in clinical trials. Pfizer invested USD 400 million (KRW 440 billion) in the clinical trial for Bapineuzumab but failed.

Aducanumab developed by Biogen/Eisai in June, 2021 was approved by the FDA over the mechanism of β-amyloid removal, however, there are still limitations to medication such as decline in blood-brain barrier permeability, inadequate therapeutic effects, side effects, high charges, etc.

Therefore, there is the highly expected need of a method and an apparatus to solve those limitations.

### [REFERENCE DOCUMENTS]

### [Patent Documents]

(Patent Document 1) Korean Patent Registration No. 10-1828954B
(Patent Document 2) Korean Patent Registration No. 10-2223507B
(Patent Document 3) Korean Patent Application No. 10-2017-7024459 (Publication No. 10-2017-0129117A)

### SUMMARY

The present disclosure aims to provide an apparatus and a method for stimulating the brain by entraining oscillation synchronized in a plurality of brain areas of an object.

Particularly, the present disclosure provides an apparatus and a method for deriving and applying an optimal stimulation by: stimulating the brain according to a typical stimulation mode determined corresponding to a condition of an object among a plurality of typical stimulation modes; and determining whether a response to entrainment of gamma oscillation synchronized in a plurality of brain areas of the object based on a measured brain response is derived or not.

The present disclosure intends to transfer a stimulation having a frequency of 30 Hz to 50 Hz to an object for entraining gamma oscillation synchronized in a plurality of brain areas of the object including prefrontal cortex (PFC) and hippocampus.

The present disclosure intends to provide a customized tACS (transcranial Alternating Current Stimulation) apparatus and a method for entraining gamma oscillation synchronized in the brain, based on real-time EEG signal monitoring.

The present disclosure intends to provide a new Alzheimer’s disease therapy through a regulatory mechanism of a gamma wave of oscillation (y-oscillation).

Imbalance in the neural network activity of neurons in the brain is a common pathological phenomenon of various brain diseases, and thus the present disclosure intends to suggest a new approach to an Alzheimer’s disease therapy and monitoring through activation of a gamma wave of oscillation (y-oscillation).

The present disclosure intends to suggest an optimal gamma entrainment parameter of a personalized transcranial alternating current stimulation (tACS) apparatus and a wearable tACS apparatus.

The present disclosure intends to suggest a “tACS+EEG all-in-one device” and an App-based EEG monitoring system.

The present disclosure intends to suggest verification of a gamma brain wave induction performance of a tACS apparatus targeting a normal aged person, and an optimal stimulation condition for brain wave entrainment different depending on an individual.

The present disclosure intends to suggest a personalized therapeutic algorithm for Alzheimer’s disease based on a neurofeedback algorithm.

The present disclosure intends to suggest methods for analyzing and representing brain stimulation and rehabilitation effects through building of a metaverse.

The present disclosure intends to suggest a system and a method for verifying and representing correlation between effects of Aβ PET (Amyloid beta PET) and cognitive function test-based gamma entrainment.

The present disclosure intends to induce a local field potential (LFP) of 40 Hz in prefrontal cortex and hippocampus of an object, based on entrainment of gamma oscillation through stimulation.

The present disclosure intends to suggest effects that a neuronal activity is regulated between brain areas of the object including prefrontal cortex and hippocampus and neural degeneration is declined, through gamma entrainment.

Further, the present disclosure intends to reduce amyloid plaques and hyperphosphorylation of tau, in brain areas of the object including prefrontal cortex and hippocampus, through entrainment of gamma oscillation.

Further, the present disclosure intends to reduce losses of neurons and synapses, brain atrophy, bulging of a ventricle in the brain and neuroinflammation, in brain areas of the object including prefrontal cortex and hippocampus through entrainment of gamma oscillation.

Further, the present disclosure intends to reduce an immune response of at least a part of microglia, to deform the microglia morphologically and to increase proteolysis in the microglia, in brain areas of the object including, prefrontal cortex and hippocampus, through entrainment of gamma oscillation.

Further, the present disclosure intends to improve abnormally transformed genes and proteins relating to at least one of membrane trafficking, intracellular trafficking, a synapse function, neuroinflammation, an apoptosis process and a DNA damage, in brain areas of the object including PFC and hippocampus through entrainment of gamma oscillation.

In the end, the present disclosure intends to provide a user with a therapy relating to AD, Parkinson’s disease, stroke, epilepsy, schizophrenia, etc.

Meanwhile, technical solutions to be achieved by the present disclosure are not limited to the aforementioned suggestions, and other not-mentioned technical solutions may be clearly understood by the skilled person in the art to which the present disclosure pertains from the description below.

According to a first embodiment of the present disclosure, a customized tACS(transcranial Alternating Current Stimulation) apparatus for stimulating a brain wave by entraining synchronized oscillation based on real-time EEG signal monitoring may include: a stimulation part that stimulates a brain of an object; a sensing part that measures a response of the brain to a stimulation; and a control part that determines whether a first response entraining oscillation synchronized in a plurality of brain areas of the object is derived or not, based on a measured brain response. The stimulation part may be an electrical stimulation part that transfers a transcranial alternating current stimulation (tACS), and the transcranial alternating current stimulation may be a first combined stimulation in which a signal is repeatedly ON/ OFF according to a predetermined first frequency and an ON signal according to the first frequency is applied as a stimulation according to a predetermined second frequency. The control part may process a first signal by the first combine stimulation among signals measured by the sensing part as a noise, and may determine whether the first response is derived or not, based on a second signal except the first combined stimulation among the signals measured the sensing part. When the first response is not derived according to the first combined stimulation, the control part may control the electrical stimulation part to transfer, to the brain of the object, a second combined stimulation resulting from correction of at least one of the first frequency, the second frequency and an output, a waveform and a of a stimulation according to the second frequency.

Further, the first frequency may be applied to induce entrainment of oscillation synchronized in the polarity of brain areas of the object, the second frequency may be applied to induce a membrane action potential and brain oscillation in the polarity of brain areas of the object, and the second frequency may have a higher value that the first frequency.

Further, the stimulation part may stimulate the brain of the object according to a stimulation mode determined corresponding to a condition of the object, the condition of the object denotes a disease relating to the object and a progressive degree of the disease, and the disease may include Alzheimer’s disease, Parkinson’s disease, stroke, epilepsy and schizophrenia.

Further, the first response may be about entrainment of γ-oscillation synchronized in the plurality of brain areas of the object, and the first frequency may be a frequency of 30 Hz to 50 Hz for entraining the gamma oscillation synchronized in the plurality of brain areas of the object including prefrontal cortex (PFC) and hippocampus.

Further, an interference signal by the first combined stimulation may be ignored based on that a magnitude of the first signal has a difference of a predetermined numeric value or more as compared to a magnitude of the second signal.

Further, the sensing part may be a brain wave measurement part that measures a brain wave of the brain induced corresponding to the stimulation, and the control part may determine whether the first response is derived or not by using a determination of whether a measured brain wave corresponds to an output and a wave form of a entrained brain wave that appears only when entraining the gamma oscillation or not.

The control part may calculate an average of power spectrum values of respective frequency bands and a standard deviation thereof, and a ratio of each average value according to at least one brain wave combinations of gamma (γ)/ alpha (α)/ beta (β)/ delta (δ)/theta (θ) brain waves from the measured brain wave, thus extracting a property of the measured brain wave.

Further, when the first response is not derived according to the second combined stimulation, the control part may control the electrical part to transfer, to the brain of the object, a third combined stimulation resulting from additional correction of at least one of the first frequency, the second frequency and an output, a waveform and a period of a stimulation according to the second frequency.

Further, when the first response is not derived according to the first combined stimulation, the control part may control the second combined stimulation according to a 1-1 frequency that is lower than the first frequency of the first combined stimulation to be transferred to the brain of the object. When the first response is not derived according to the second combined stimulation, the control part may control the third combined stimulation according to a 1-2 frequency that is lower than the 1-1 frequency to be transferred to the brain of the object, and the 1-1 frequency and the 1-2 frequency may be correctable to less than 30 Hz.

Further, a local field potential (LFP) of 40 Hz may be induced in the prefrontal cortex and the hippocampus of the object through entrainment of the gamma oscillation through stimulation.

Further, through entrainment of the gamma oscillation, a neuronal activity may be regulated among the brain areas of the object including the prefrontal cortex and the hippocampus, and a balance of transmission between GABAergic and glutamatergic neurons that are secreted by excitatory and inhibitory activities of the neuron may be induced.

Further, through entrainment of the gamma oscillation, amyloid plaques may be reduced in the brain areas of the object including the prefrontal cortex and the hippocampus, and hyperphosphorylation of tau may be reduced in the brain areas of the object including the prefrontal cortex and the hippocampus.

Further, through entrainment of the gamma oscillation, losses of neurons and synapses may be reduced in the brain areas of the object including the prefrontal cortex and the hippocampus, brain atrophy may be reduced in the brain areas of the object including the prefrontal cortex and the hippocampus, atrophy may be reduced in the brain areas of the object including the prefrontal cortex and the hippocampus, bulging of a ventricle in the brain may be reduced in the brain areas of the object including the prefrontal cortex and the hippocampus, and neuroinflammation may be reduced in the brain areas of the object including the prefrontal cortex and the hippocampus.

Further, through entrainment of the gamma oscillation, an immune response of at least a part of microglia may be reduce in the brain areas of the object including the prefrontal cortex and the hippocampus, the microglia may be deformed morphologically in the brain areas of the object including the prefrontal cortex and the hippocampus, and proteolysis in at least a part of the microglia may be increased in the brain areas of the object including prefrontal cortex and hippocampus.

Meanwhile, according to a second embodiment of the present disclosure, a system may include: an apparatus; and a server forming a network with the apparatus, wherein the apparatus may include: a stimulation part that stimulates a brain of an object; a sensing part that measures a response of the brain to a stimulation; and a communication part that transmit a measured response of the brain to the server. The server may transmit a first information for the measured response of the brain to a predetermined outside, receives, from the outside, a second information for determination of whether a first response entraining gamma oscillation synchronized in a plurality of brain areas of the object is derived or not based on the measured response of the brain, and transmit the second information to the communication part of the apparatus. The stimulation part of the apparatus may be an electrical stimulation part that transfers a transcranial alternating current stimulation (tACS) and the transcranial alternating current stimulation may be a first combined stimulation in which a signal is repeatedly ON/ OFF according to a predetermined first frequency and an ON signal according to the first frequency is applied as a stimulation according to a predetermined second frequency. The outside may process a first signal by the first combine stimulation among signals measured by the sensing part as a noise, and determines whether the first response is derived or not, based on a second signal except the first combined stimulation among the signals measured the sensing part. When the first response is not derived according to the first combined stimulation based on the second information, the electrical stimulation part may transfer, to the brain of the object, a second combined stimulation resulting from correction of at least one of the first frequency, the second frequency and an output, a waveform and a period of a stimulation according to the second frequency.

Meanwhile, according a third embodiment of the present disclosure, a system may include: an apparatus; and a server forming a network with the apparatus, wherein the apparatus may include: a stimulation part that stimulates a brain of an object; a sensing part that measures a response of the brain to a stimulation; a control part that determines whether a first response entraining oscillation synchronized in a plurality of brain areas of the object is derived or not, based on a measured brain response; and a communication part that transmit a measured response of the brain to the server. The server may transmit a first information for the measured response of the brain to a predetermined outside, receives, from the outside, a second information for determination of whether a first response entraining gamma oscillation synchronized in a plurality of brain areas of the object is derived or not based on the measured response of the brain, and transmit the second information to the communication part of the apparatus. The stimulation part of the apparatus may be an electrical stimulation part that transfers a transcranial alternating current stimulation (tACS) and the transcranial alternating current stimulation may be a first combined stimulation in which a signal is repeatedly ON/ OFF according to a predetermined first frequency and an ON signal according to the first frequency is applied as a stimulation according to a predetermined second frequency. The outside may process a first signal by the first combine stimulation among signals measured by the sensing part as a noise, and determine whether the first response is derived or not, based on a second signal except the first combined stimulation among the signals measured the sensing part. When the first response is not derived according to the first combined stimulation by using a first determination and a second determination together, the control part may control the electrical stimulation part to transfer, to the brain of the object, a second combined stimulation resulting from correction of at least one of the first frequency, the second frequency and an output, a waveform and a period of a stimulation according to the second frequency.

### Advantageous Effect

According to one embodiment of the present disclosure, it is capable of providing an apparatus and a method for stimulating the brain by entraining oscillation synchronized in a plurality of brain areas of an object.

Particularly, the present disclosure is capable of providing an apparatus and a method for deriving and applying an optimal stimulation by: stimulating the brain according to a typical stimulation mode determined corresponding to a condition of an object among a plurality of typical stimulation modes; and determining whether a response to entrainment of gamma oscillation synchronized in a plurality of brain areas of the object based on a measured brain response is derived or not.

The present disclosure is capable of transferring a stimulation having a frequency of 30 Hz to 50 Hz to an object for entraining gamma oscillation synchronized in a plurality of brain areas of the object including prefrontal cortex (PFC) and hippocampus.

The present disclosure is capable of providing a customized transcranial alternating current stimulation apparatus and a method for entraining gamma oscillation synchronized in the brain, based on real-time EEG signal monitoring.

The present disclosure is capable of providing a new Alzheimer’s disease therapy through a regulatory mechanism of a gamma wave of oscillation (y-oscillation).

Imbalance in the neural network activity of neurons in the brain is a common pathological phenomenon of various brain diseases, and thus the present disclosure is capable of providing a new approach to an Alzheimer’s disease therapy and monitoring through activation of a gamma wave of oscillation (y-oscillation).

The present disclosure is capable of providing an optimal gamma entrainment parameter of a personalized transcranial alternating current stimulation (tACS) apparatus and a wearable tACS apparatus.

The present disclosure is capable of providing a “tACS+EEG all-in-one device” and an App-based EEG monitoring system.

The present disclosure is capable of providing verification of a gamma brain wave induction performance of a tACS apparatus targeting a normal aged person, and an optimal stimulation condition for brain wave entrainment different depending on an individual.

The present disclosure is capable of providing a personalized therapeutic algorithm for Alzheimer’s disease based on a neurofeedback algorithm.

The present disclosure is capable of providing methods for analyzing and representing brain stimulation and rehabilitation effects through building of a metaverse.

The present disclosure is capable of providing a system and a method for verifying and representing correlation between effects of Aβ PET (Amyloid beta PET) and cognitive function test-based gamma entrainment.

The present disclosure is capable of inducing a local field potential (LFP) of 40 Hz in prefrontal cortex and hippocampus of an object, based on entrainment of gamma oscillation through stimulation.

Further, the present disclosure is capable of providing effects that a neuronal activity is regulated between brain areas of the object including prefrontal cortex and hippocampus and neural degeneration is declined, through gamma entrainment.

Further, the present disclosure is capable of reducing amyloid plaques and hyperphosphorylation of tau, in brain areas of the object including prefrontal cortex and hippocampus, through entrainment of gamma oscillation.

Further, the present disclosure is capable of reducing losses of neurons and synapses, brain atrophy, bulging of a ventricle in the brain and neuroinflammation, in brain areas of the object including prefrontal cortex and hippocampus through entrainment of gamma oscillation.

Further, the present disclosure is capable of reducing an immune response of at least a part of microglia, of deforming the microglia morphologically and of increasing proteolysis in the microglia, in brain areas of the object including, prefrontal cortex and hippocampus, through entrainment of gamma oscillation.

Further, the present disclosure is capable of improving abnormally transformed genes and proteins relating to at least one of membrane trafficking, intracellular trafficking, a synapse function, neuroinflammation, an apoptosis process and a DNA damage, in brain areas of the object including prefrontal cortex and hippocampus through entrainment of gamma oscillation.

In the end, the present disclosure is capable of providing a user with the therapy relating to AD, Parkinson’s disease, stroke, epilepsy, schizophrenia, etc.

Meanwhile, advantageous effects to be obtained by the present disclosure are not limited to the aforementioned effects, and other not-mentioned advantageous effects may be clearly understood by the skilled person in the art to which the present disclosure pertains from the description below.

### BRIEF DESCRIPTION OF THE DRAWINGS

The accompanying drawings of this specification exemplify a preferred embodiment of the present disclosure, and helps more clearly understanding the spirit of the present disclosure along with the following detailed description. Thus, it will be understood that the present disclosure is not limited to only contents illustrated in the accompanying drawings;

FIG. 1 shows a block view of an apparatus for stimulating the brain by entraining oscillation synchronized in a plurality of brain areas of an object, according to the present disclosure,

FIGS. 2 and 3 show a view explaining stimulation modes relating to the present disclosure,

FIG. 4 shows a view for explaining a mechanism of an Alzheimer’s disease therapy through entrainment of gamma wave of oscillation (y-oscillation) according to the present disclosure,

FIG. 5 show a view for explaining an advantageous effect of a tACS that is the most suitable for entrainment of gamma wave of oscillation, in accordance with the present disclosure,

FIG. 6 shows a view for explaining an apparatus that senses a tACS and a brain wave,

FIG. 7 shows a view for explaining an EEG-based closed loop neuro-feedback tACS mode suggested in the present disclosure,

FIG. 8 shows one example of a diagrammatic view for development of a tACS+EEG all-in-one device, in accordance with the present disclosure,

FIG. 9 shows one example of a block view of a real-time tACS-EEG Neurofeedback algorithm, in accordance with the present disclosure,

FIG. 10 shows one example of derivation and visualization of a correlation between an Aβ PET (Amyloid beta PET) image and a gamma band, in accordance with the present disclosure,

FIG. 11 shows one example of a therapeutic system through App-based EEG monitoring and cooperation with an external medical institute, in accordance with the present disclosure,

FIGS. 12A and 12B show test results for explaining reduction of amyloid-β protein and improvement in a cognitive function by inducing entrainment of gamma oscillation, in accordance with the present disclosure,

FIG. 13 shows a test result for explaining induction of a conformational change of the brain and improvement of a cognitive function of a patient with Alzheimer’s disease through gamma oscillation entrainment therapy, in accordance with the present disclosure,

FIGS. 14A and 14B show views explaining a relation between a microglial activity and tau tangles, in accordance with the present disclosure, and FIG. 15 shows a view for explaining improvement of tau tangles by regulating a microglial activity through gamma oscillation entrainment therapy, and

FIG. 16 shows a view for explaining expandability of the therapeutic field to nervous system diseases through equivalence verification of a product according to the present disclosure, in accordance with the present disclosure.

### DETAILED DESCRIPTION OF THE PREFERRED EMBODIMENT

Hereinafter, exemplary embodiments of the present disclosure will be described in detail so as to be easily implemented by the skilled person in the art, with reference to the accompanying drawings. A description of the present disclosure is merely an exemplary embodiment for a structural or functional description and the scope of the present disclosure should not be construed as being limited by exemplary embodiments described in a text. That is, since the exemplary embodiment can be variously changed and have various forms, the scope of the present disclosure should be understood to include equivalents capable of realizing the technical spirit. Further, it should be understood that since a specific exemplary embodiment should not include all objects or effects or include only the effect, the scope of the present disclosure is not limited by the object or effect.

Meanings of terms described in the present disclosure should be understood as follows.

The terms “first”, “second”, and the like are used to differentiate a certain component from other components, but the scope of the rights should not be construed to be limited by the terms. For example, a first component may be referred to as a second component, and similarly, the second component may be referred to as the first component. It should be understood that, when it is described that a component is “connected to” the other component, the component may be directly connected to the other component or another component may be present therebetween. In contrast, it should be understood that when it is described that a component is “directly connected to” the other component, another component is not present therebetween. Meanwhile, other expressions describing the relationship between the components, that is, expressions such as “between” and “directly between” or “adjacent to” and “directly adjacent to” should be similarly interpreted.

It is to be understood that the singular expression encompasses a plurality of expressions unless the context clearly dictates otherwise and it should be understood that the term “including” or “having” indicates that a feature, a number, a step, an operation, a component, a part, or the combination thereof described in the specification is present, but does not exclude a possibility of presence or addition of one or more other features, numbers, steps, operations, components, parts or combinations thereof, in advance. Unless otherwise a singular form has a explicitly different meaning contextually,

If it is not contrarily defined, all terms used herein have the same meanings as those generally understood by the skilled person in the art. Terms which are defined in a generally used dictionary should be interpreted to have the same meaning as the meaning in the context of the related art, and are not interpreted as an ideal meaning or excessively formal meanings unless clearly defined in the present disclosure.

### Apparatus Stimulating the Brain by Entraining γ-Oscillation Synchronized in Brain Areas

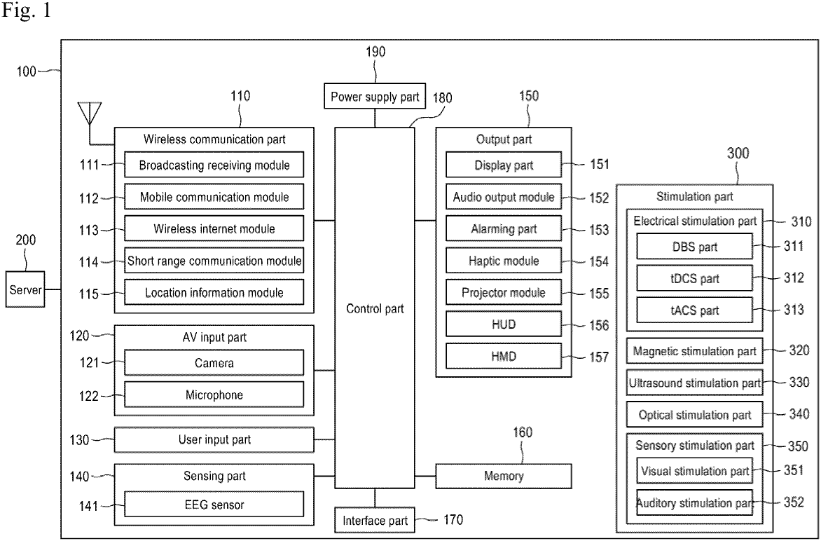

FIG. 1 shows one example of a block view of a brain stimulation system, in accordance with the present disclosure.

Referring to FIG. 1, a brain stimulation system (1) may include a brain stimulation device 100 and a server 200.

The Server 200 may form a network with a medical institute, receiving information relating to medical personnel’s determination and opinion, and transferring the received information to the brain stimulation device 100.

Firstly, the brain stimulation device 100 may include a wireless communication part 110, an AV (Audio/Video) input part 120, a user input part 130, a sensing part 140, an output part 150, a memory 160, an interface part 170, a control part 180 and a power supply part 190, a stimulation part 300, etc.

However, configurational elements illustrated in FIG. 1 are not essential, and thus the brain stimulation system 1 composed of more or less configurational elements than those may be implemented.

Hereinafter, the configurational elements are described one by one.

The wireless communication part 110 may include at least one module that allows a wireless communication in between the brain stimulation system 1 and a wireless communication system or an apparatus and a network wherein the apparatus is placed.

For example, the wireless communication part 110 may include a mobile communication module 112, a wireless internet module 113, a short range communication module 114, a location information module 115, etc.

A broadcasting receiving module 111 receives a broadcasting signal and/or broadcasting-related information from an external broadcasting management server through a broadcasting channel.

The broadcasting channel may include a satellite channel and an on-air channel. The broadcasting management server may signify a server that generates and transmits a broadcasting signal and/or broadcasting-related information, alternatively signifying a server that receives a pre-generated broadcasting signal and/ or broadcasting related information and transmits this to the brain stimulation device 100. The broadcasting signal may include a TV broadcasting signal, a radio broadcasting signal, a data broadcasting signal, as well as a broadcasting signal in a combination form of the preceding.

The broadcasting-related information may signify information relating to a broadcasting channel, a broadcasting program or a broadcasting service provider. The broadcasting-related information may be also provided through a mobile communication network. In such a case, this may be received by the mobile communication module 112.

The broadcasting-related information may exist in various forms, for example, EPG (Electronic Program Guide) of DMB (Digital Multimedia Broadcasting) or EGS (Electronic Service Guide) of DVB-H (Digital Video Broadcast-Handled), etc.

The broadcasting receiving module 111 may receive a digital broadcasting signal by using a digital broadcasting system, such as DMB-T (Digital Multimedia Broadcasting-Terrestrial), DMB-S (Digital Multimedia Broadcasting-Satellite), MediaFLO (Media Forward Link Only), DVB-H (Digital Video Broadcast-Handheld), DVB-CBMS, OMA-BCAST, ISDB-T (Integrated Services Digital Broadcast-Terrestrial), etc. Further, the broadcasting receiving module 111 may be configured to be suitable for the aforementioned digital broadcasting system as well as other broadcasting systems.

The broadcasting signal and/ or broadcasting-related information that were received through the broadcasting receiving module 111 may be stored in the memory 160.

The mobile communication module 112 transmits and receives a wireless signal with at least one of a base station, the external brain stimulation device 100 and a server on a mobile communication network. The wireless signal may include various forms of data according to transmitting and receiving of a speech call signal, a vide call signal or a text/multimedia message.

The wireless internet module 113 refers to a module for a wireless internet connection, and may be built in or on the exterior of the brain stimulation device 100.

Wireless WLAN (Wireless LAN) (Wi-Fi), Wibro (Wireless broadband), Wimax (World Interoperability for Microwave Access), HSDPA (High Speed Downlink Packet Access), etc. may be used as a technology for the aforementioned wireless internet.

The short range communication module 114 refers to a module for short range communication. Bluetooth, RFID (Radio Frequency Identification), IrDA (infrared Data Association), UWB (Ultra-Wideband), ZigBee, etc. may be used as a technology for the short range communication.

The position information module 115 refers to a module for acquiring a position of the brain stimulation device 100 and a representative example thereof is a GPS (Global Position System) module. According to the modern technology, the GPS module 115 calculates distance information away from at least three satellites and an accurate time information and then applies trigonometry to the calculated information, allowing accurate calculation of three dimensional position information depending on latitude, longitude and altitude. Currently, widely used is a method to calculate position and time information using three satellites and to calibrate an error on the calculated position and time information using one more satellite. Further, the GPS module 115 continuously calculates a current position in real time to calculate velocity information.

Referring to FIG. 1, the AV input part 120 is for inputting an audio signal or a video signal and may include a camera 121, a microphone 122, etc.

The camera 121 processes an image frame such as a still image, a movie or etc. that was obtained by an image sensor on the photographing mode and may represent a processed image frame on a display part 151.

The processed image frame in the camera 121 may be stored in a memory 160 or transmitted to the outside through the wireless communication part 110.

At least two cameras 121 may be provided according to a user environment.

The microphone 122 receives an input external audio signal by a microphone on recording mode, speech recognition mode, etc. and processes this signal to electrical speech data.

The processed speech data is converted into a form that is transmittable to a mobile communication base station through the mobile communication module 112, and then may be output.

Various noise removal algorithms for removing noises that occur during the receipt of the input external audio signal may be implemented in the microphone 122.

Next, the user input part 130 generates input data for a user to control operation of the brain stimulation system 1. The user input part 130 may be composed of a key pad, a dome switch, a touch pad (static pressure/static electricity), a jog wheel, a jog switch, etc.

The sensing part 140 generates a sensing signal for controlling the operation of the brain stimulation system 1 by sensing a current state of the brain stimulation system 1, such as an open and close state of the brain stimulation system 1, a position of the brain stimulation system 1, user’s touch or not, a bearing of the brain stimulation system 1, acceleration/ deceleration of the brain stimulation system 1, etc.

The sensing part 140 may sense power supply of the power supply part 190 or not, connection of the interface part 170 to an external apparatus or not, etc.

Particularly, the sensing part 140 according to the present disclosure may include a brain wave measurement part 141.

A brain wave is generated by an object’s thinking and emotions, while behaviors are generated by communication between neurons existing in the brain. The brain wave refers to a synchronized electrical wave that is generated when neurons of the cerebral cortex. Brain waves may be measured through electroencephalogram (EEG) that measures an electric potential difference between surface electrodes positioned at certain regions of the scalp. The brain wave shown by EEG is the sum of electrical activities of multitudinous neurons of the cerebral cortex underlying the surface electrodes.

Brain waves appear at various frequency bands, and theses frequency bands show states of the brain. The brain waves are sorted into a delta (δ) wave, a theta (θ) wave, an alpha (α) wave, a beta (β) wave, a gamma (γ) wave, etc.

The delta (δ) wave has a frequency band of less than 4 Hz and a broad amplitude. This refers to a wave form appearing during a deep sleep state in which no dreaming occurs.

The theta (θ) wave refers to a brain wave of 4 to 7 Hz and occurs during a particular sleep state. This is a wave form that also occurs during deep meditation. It is known that the theta (θ) wave is involved in solidifying memories resulting from learning during the sleep.

The an alpha (α) wave is approximately 8 to 13 Hz, referring to a wave occurring in a state of consciousness taking a rest.

The beta (β) wave is 14 to 29 Hz, referring to a rhythm of activated cerebral cortex. This is a wave form that occurs when the cerebral cortex performs a general cognitive activity in a state of consciousness.

The gamma (γ) wave is a wave form of 30 to 80 Hz, referring to high frequency brain wave that occurs in a state of either tension or excitement. This is known as a wave form occurring in a state of high concentration.

EEG is a non-invasive technique to measure a brain wave, measuring a potential difference between two electrodes following fixing planar electrodes to the scalp using an electrically conductive glue.

Several electrodes are attached to standard positions on the scalp and a voltage change having a weak amplitude, generated from neurons in the cerebral cortex is amplified and recorded by a brain wave recorder.

The brain wave appears through a synapse current occurring when neurons in the cerebral cortex positioning immediately below the cranium communicate with each other.

A synapse current of one neuron is extremely weak and should pass several layers of a brain membrane, cerebrospinal fluid, the cranium and a scalp layer in order for a signal thereof to reach an electrode attached to the scalp. The reason why electrical record of a brain wave is possible is that a brain wave is the sum of signals generated when thousands of neurons are activated all together. Therefore, as activities of the neurons are synchronized more, a large amplitude of brain wave in a form of a low frequency appears.

Meanwhile, the output part 150 is to generate an output relating to a sense of sight, hearing, touch or etc. and thus may include a display part 151, an audio output module 152, an alarming part 153, a haptic module 154 and a projector module 155, a head-up display (HUD), a head mounted display (HMD), etc.

The display part 151 represents (outputs) information that was processed in the brain stimulation system 1.

The display part 151 may include at least one of a liquid crystal display (LCD), a thin film transistor-liquid crystal display (TFT LCD), an organic light-emitting diode (OLED), a flexible display, a 3D display, etc.

A part of these displays may be formed into a transparent type or a light transmissive type, thus allowing seeing the outside therethrough. This refers to a transparent display and a representative example thereof is a transparent OLED(TOLED), etc. A rear structure of the display part 151 may be also formed into a light transmissive type structure. Due to this structure, a user may see an object positioned on the rear of the brain stimulation system 1 through an area occupied by the display part 151 in the body of the brain stimulation system 1.

At least two display parts 151 may exist, depending on an implement of the brain stimulation system 1. For example, a plurality of display parts may be positioned apart from each other or in a line on one side in the brain stimulation system 1. Further, these may be positioned on different sides, respectively.

When the display part 151 and a sensor that senses a touch action (‘touch sensor’) mutually form a layer structure (‘touch screen’), the display part 151 may be used as an input device besides an output device. The touch sensor may have a form, for example, a touch film, a touch sheet, a touch pad, etc.

The touch sensor may be configured to convert a change in a pressure applied to a specific region of the display part 151 or static electricity occurring in a specific region of the display part 151, etc. into an electrical input signal. The touch sensor may sense touched position and area as well as an input when touched.

When a touch input for the touch sensor occurs, signal(s) corresponding thereto is sent to a touch controller. The touch controller processes those signal(s) and then transmits relevant data to the control part 180. The control part 180 hereby sees what area of the display part 151 was touched is.

A proximity sensor 141 may be positioned in an internal area of the brain stimulation system 1 that was enclosed with the touch screen, or in the vicinity of the touch screen. The proximity sensor refers to a sensor that detects existence or nonexistence of an object approaching a predetermined detecting side or an object in the vicinity by using an electromagnetic force or an infrared ray without any mechanical contact. The lifespan of the proximity sensor is longer than a contact typed sensor and the utilization thereof is also high.

Examples of the proximity sensor include a transmission type of photoelectric sensor, a direct reflection type of photoelectric sensor, a mirror reflection type of photoelectric sensor, a high frequency oscillation type of proximity sensor, a capacitive proximity sensor, a magnetic proximity sensor, an infrared proximity sensor, etc. When the touch screen is an electrostatic type, this is configured to detect proximity of a pointer by a change in an electric field depending on the pointer’s approaching. In this case, the touch screen (touch sensor) may be classified as a proximity sensor.

The proximity sensor senses a proximity touch and a proximity touch pattern (for example, a proximity touch distance, a proximity touch direction, a proximity touch speed, a proximity touch time, a proximity touch position, a proximity touch movement, etc.). Information corresponding to the sensed proximity touch action and proximity touch pattern may be output on the touch screen.

The audio output module 152 may output audio data that was received from the wireless communication part 110 on recording mode, speech recognition mode, broadcasting receiving mode, etc. or stored in the memory 160.

The audio output module 152 outputs an audio signal relating to a function performed in the brain stimulation system 1. This audio output module 152 may include a receiver, a speaker, a buzzer, etc.

The alarming part 153 may output a signal for notifying occurrence of events in the brain stimulation system 1.

The alarming part 153 may output a signal for notifying occurrence of those events in a different form, for example, a vibration, besides a video signal or an audio signal.

The video signal or audio signal may be output through the display part 151 or the speech output module 152. Thus, these 151, 152 may be classified as a part of the alarming part 153.

The haptic module 154 generates various tactile effects which a user can feel. A representative example of tactile effects generated from the haptic module 154 is a vibration. An intensity, a pattern, etc. of the vibration generated from the haptic module 154 may be controlled.

For example, different vibrations may be synthesized and output, or may be output successively.

Besides a vibration, the haptic module 154 may generate various tactile effects including an effect resulting from a stimulation such as spray injection power or suction power through a pin arrangement that vertically moves with respect to a contacted skin surface, an spray injection port or a suction port, brush on a skin surface, contact of an electrode, electrostatic force, etc., an effect resulting from recreation of cold/warm feelings using elements capable of absorbing or generating heat, etc.

The haptic module 154 may be implemented to transmit a tactile effect through direct contact as well as muscular senses of user’s fingers and arms. At least two haptic modules 154 may be provided according to an aspect of the present disclosure.

The projector module 155 is a configuration element for performing an image project function and may display, on an external screen or wall, an image which is the same as or at least part of which an image represented on the display part 151 according to the control part 180, or at least a part of which is different therefrom.

Particularly, the projector module 155 may include a light source (not illustrated) that generates light for outputting an image to the outside (for example, a laser light beam), an image generating unit (not illustrated) that generates an image to be output to the outside using the light generated from the light source, and a lens (not illustrated) that enlarges and outputs the image at a predetermined focal distance to the outside. Further, the projector module 155 may include a device (not illustrated) capable of adjusting an image projection direction by mechanically moving the lens or the entire module.

The projector module 155 may be classified into a CRT (Cathode Ray Tube) module, a LCD (Liquid Crystal Display) module, a DLP (Digital Light Processing) module, etc. according to the type of an element of the display unit. Especially, the DLP module may be advantageous in miniaturization of the projector module 151 in a way to enlarge and project the image generated by reflecting the light generated from the light source on a DMD (Digital Micromirror Device) chip.

Preferably, the projector module 155 may be provided at a side, a front side or a rear side of the brain stimulation system 1 in a longitudinal direction. Definitely, the projector module 155 may be also provided at any position of the brain stimulation system 1, if necessary.

Further, a head-up display(HUD) 156 signifies a device that projects information for a current vehicle speed, a residual fuel amount, a navigation guide, etc. into a graphic image on the window in front of a driver in a vehicle.

Further, a head mounted display(HMS) 157 is a representative device that outputs virtual reality information.

Virtual reality is a generic term for an interface between a human and a computer, which prepares a 3D content for a certain environment or circumstance to make a user of the 3D content feel like interacting with a real surrounding circumstances or environment.

Generally, a three-dimensional effect perceived by a person results from combination of a degree of a change in the thickness of a crystalline lens, a difference in an angle between both eyes and an object, differences in the positon and shape of the object shown in right and left eyes, a time difference according to the movement of the object, effects of various mentalities and memories, etc.

The most key factor making a person feel a three dimensional effect is a binocular disparity appearing when both eyes of the person apart about 6.5 cm in a transverse direction. That is, the person looks at an object with an angle difference caused by a time difference between both eyes, this difference resulting in different images come into the respective eyes. Two images are transmitted to a brain through a retina, the brain amalgamating information for the two images accurately to allow the person to feel a 3D image.

Such a 3D content has been widely used in the field of various media already and received a favorable review from consumers. For example, a 3D movie, a 3D game and an experience-display are representative.

It is diversely demanded to popularize a 3D content according to the virtual reality technology and to develop a technology for providing virtual reality services accompanied with higher-level immersion of a user.

Generally, an image display device refers to an image-representing device that forms a focus to form a virtual big-screen at a far distance with an image light generated at a very proximate position to eyes by using a precise optical device, thus allowing a user to see an enlarged virtual image.

Further, the image display device may be sorted into a see-close type allowing a user to see not a surrounding environment but an image light emitted from a display element only, and a see-through type allowing the user to see the surrounding environment through a window and the image light emitted from the display element at the same time.

The HMD 157 refers to any of various digital devices which allows a user to receive a multimedia content by wearing on the head like glasses. According to current trends to lighten the weight of and to miniaturize a digital device, various wearable computers are developed and HMDs widely used are.

For example, when a microphone and a speaker are mounted on the HMD 157, a user wearing the HMD 157 may have a phone call. Further, for example, when an infrared camera 122 is mounted on the HMD 157, the user wearing the HMD 157 may capture a desired direction of image.

Further, the memory 160 may store a program for processing and controlling the control part 180 and perform a function for temporarily storing input/output data (for example, a text message, an audio, a still image, a movie, etc.). The memory 160 may also store a usage frequency for the respective data. Further, the memory 160 may store data relating to various patterns of a vibration and an audio that were output when performing a touch input on the touch screen.

The memory 160 may include at least one storing medium selected from a flash memory, a hard disk type of memory, a multimedia card micro type of memory, a card type of memory(for example, SD or XD memory, etc.), a Random Access Memory(RAM), a Static Random Access Memory(SRAM), a Read-Only Memory(ROM), an Electrically Erasable Programmable Read-Only Memory(EEPROM), a Programmable Read-Only Memory(PROM), a magnetic memory, a magnetic disc and an optical disc. The brain stimulation system 1 may be operated in association with a web storage that performs a storing function of the memory 160 on the internet.

The interface part 170 serves as a connecting passage to all external apparatuses that are connected to the brain stimulation system 1. The interface 170 receives data or power from an external apparatus to transmit the received data or power to respective configuration elements inside the brain stimulation system 1 or to transmit data inside the brain stimulation system 1 to the external apparatus. For example, the interface part 170 may include a wire/wireless headset port, an external charger port, a wire/wireless data port, a memory card port, a port connecting a device having an identification module, an audio Input/Output(I/O) port, a video Input/Output(I/O) port, an earphone port, etc.

The identification module is a chip that stores various kinds of information for authenticating the use of the brain stimulation system 1, and may include a User Identify Module (UIM), a Subscriber Identify Module (SIM), a Universal Subscriber Identity Module (USIM), etc. A device having the identification module (‘identification device’) may be manufactured into a smart card type. Thus, the identification device may be connected with the brain stimulation system 1 through a port.

The interface part may be a passage for supplying power from external cradles to the brain stimulation system 1 when the brain stimulation system 1 is connected to the cradles, or a passage for transmitting various command signals that were input from the cradles by a user to the mobile apparatus. The various command signals or power that were input from the cradles may serve as a signal for recognizing whether the mobile apparatus is accurately mounted on the cradles or not.

The control part 180 generally controls overall operation of the brain stimulation system 1.

The power supply part 190 receives applied external power and internal power to supply power required for operating respective configuration elements, by controlling by the control part 180.

Various embodiments described herein may be implemented in a recoding medium that is readable by a computer or a device similar thereto, for example, by using a software, a hardware or a combination of the preceding.

According to an implementation in aspect of hardware, an embodiment described herein may be implemented by using at least one of ASICs (Application Specific Integrated Circuits), DSPs(Digital Signal Processors), DSPDs (Digital Signal Processing Devices), PLDs (Programmable Logic Devices), FPGAs (Field Programmable Gate Arrays), processors, controllers, micro-controllers, microprocessors and electrical units for performing other functions. In some cases, embodiments described herein may be implemented as the control part 180 itself.

According to an implementation in aspect of software, embodiments, such as procedures and functions, described herein may be implemented as separate software modules. The respective software modules may perform one or more functions and operations described herein. A software code may be implemented with a software application written in an appropriate programming language. The software code is stored in the memory 160 and may be performed by the control part 180.

Further, the stimulation part 300 provides a function that stimulates the brain of a user through a neuromodulation technique, a neurofeedback technique, a sensory stimulation technique, etc.

The stimulation part 300 according to the present disclosure may include an electrical stimulation part 310, a magnetic stimulation part 320, an ultrasound stimulation part 330, an optical stimulation part 340 and a sensory stimulation part 350.

Firstly, the electrical stimulation part 310 may include a DBS part 311, a tDCS part 312 and a tACS part 313.

The DBS part 311 uses a deep brain stimulation, stimulating activities of neurons by positioning a microelectrode in a deep brain nuclei region.

A DBS (deep brain stimulation) may be provided in a DC type, a Pulse type and an NIR type.

Regarding the deep brain stimulation, when applying a stimulation to a nucleus of a specific brain region, this disturbs a pathological signal occurring in the brain area, promoting treatment of various diseases and amelioration of symptoms.

According to the deep brain stimulation, a microelectrode is placed in a deep nuclei region of the brain and electric power needed for activities is provided from a pulse generator inserted into the chest in a similar fashion to a pacemaker.

It, hereby is possible to block depolarization, that is, outputs of neurons positioned in the region of the electrode.

Further, the neuronal outputs are indirectly regulated through synapse suppression, that is, by activating an axon terminal having a synapse connection for the neuron in vicinity of the electrode.

Next, the tDCS part 312 uses transcranial direct current stimulation, stimulating neurons in cerebral cortex with a weak direct current by attaching an electrode to the head.

The tDCS part 312 is a noninvasive brain stimulation mode for recovering after-effects resulting from brain damage, allowing helping improvement in a brain function by regulating an active state of a neuron through an electrical stimulation.

Further, a tACS (transcranial alternating current stimulation) part 313 transmits micro currents in the quantity of less than 1mA to the cranium through an attached electrode, used in the non-pharmacological therapy for the amelioration of various kinds of pains such as anxiety, depression, insomnia, stress, headache, etc.

A tACS (transcranial alternating current stimulation) is effective for regulating microglia and is safe as using micro currents, and enables a middle-long term therapy without side effects.

Further, this is the most advanced therapy with high compatibility with existing chemotherapy that promotes and/or suppresses secretion of a hormone.

When applying a tACS (transcranial alternating current stimulation), the brain itself maintains stable DMN, allowing inducing user’s sleep and improving the quality of sleep.

Further, this improves hormones (e.g. serotonin, melatonin, GABA, etc.), allowing inducing user’s sleep and improving the quality of sleep.

Further, this stimulates a brain tissue, allowing returning a balance of neurochemicals back to the balance before stressed.

The magnetic stimulation part 320 uses a TMS (transcranial magnetic stimulation) and is referred to as a transcranial alternating current stimulation. This stimulates a neuron in the brain noninvasively by using magnetic energy.

This is effective for the treatment of neurological disorders and mental illness, such as depression.

This generates a strong magnetic field near the head with an electrically conductive magnetic coil, and then the magnetic field stimulates a neuron in transcranial cortex while passing through the cranium.

At this time, according to a rate in the magnetic field, it is allowable to increase or reduce an activity of cerebral cortex. For example, the activity is regulated by using a high frequency stimulation when the activity of the cerebral cortex is low such like depression, while using a low frequency stimulation when an activity thereof is too high such like anxiety and mania.

Further, the ultrasound stimulation part 330 uses an ultrasound for therapeutic effects, being widely used in gynecology, orthopedics, dermatology, etc.

The ultrasound signifies a sound wave having a frequency beyond a limit that a person can hear it. Generally, a sound wave which a healthy person can hear is 20 kHz and the ultrasound refers to a sound wave beyond this. The ultrasound therapy uses the ultrasound for therapeutic effects, being widely used in gynecology, orthopedics, dermatology, etc.

The ultrasound therapy may be sorted into a high intensity ultrasound of 1000 W/cm² or more and a low intensity ultrasound in a range of 10-50 W/cm². The high intensity ultrasound therapy selectively heats a tissue and is mainly used for the treatment of tumors. The low intensity ultrasound therapy heats a subcutaneous tissue and is used for musculoskeletal treatment, such as skin lifting, chondrocyte regeneration, etc. Further, the ultrasound therapy has an advantage of no skin damage, allowing facilitating the recovery.

HIFU (High Intensity Focused Ultrasound) therapy induces tumor necrosis or reduces tumors largely by using a heat and energy generated when focusing ultrasound beams transmitted multi-directionally, without an incision or a surgical operation. This is used for the treatment of uterine myoma, prostate cancer, cancer bone metastasis, liver cancer, etc.

LIFU (Low Intensity Focused Ultrasound) therapy secures a skin lifting effect through the necrosis of subcutaneous tissues by using a heat. A therapeutic mechanism thereof is similar to that of the HIFU but there are differences in intensities and ranges between the LIFU and the HIFU.

LIPUS (Low Intensity Pulsed Ultrasound) irradiates an ultrasound to a cure part and is used for treatments for bone fractures, chondrocyte regeneration, etc. in a way activates cells by stimulating physical oscillation.

Sonophoresis is used for transferring a drug to the skin using a low frequency of ultrasound.

Further, the optical stimulation part 340 irradiates light to the head to stimulate the brain, allowing applying a brain photo modulation mode.

According to the optical stimulation part 340, a light of 600-1000 mn invades a cell wall, being involved in COX (cytochrome oxidase) of the mitochondrial respiration chain.

There, hereby are increases in synapse generation (↑), blood flow (↑) and SOD (↑), prevention of inflammation and apoptosis, and a decrease in excite-toxicity (↓).

Further, the sensory stimulation part 350 may include a visual stimulation part 351 and an auditory stimulation part 352.

The sensory stimulation part 350 indirectly stimulates the brain by applying a stimulation to other organ rather than directly to the brain.

The visual stimulation 351 signifies an emanation from light in which a stimulation is come in a naked eye, resulting in a light sense.

For example, the visual stimulation part 351 conventionally performs full-field irradiation by using a white stroboscope flash having 100 thousands of luminous intensity with fixing a stroboscope valve 20 cm before and after the eye of an examinee in a state of closing eyes. The stimulation is performed by applying a stimulation in the order from a low frequency stimulation to a high frequency stimulation for 10 seconds, observing a brain wave or an examinee for the next 10 seconds, and then proceeding with a next step.

### Stimulation Mode Relating to the Present Disclosure

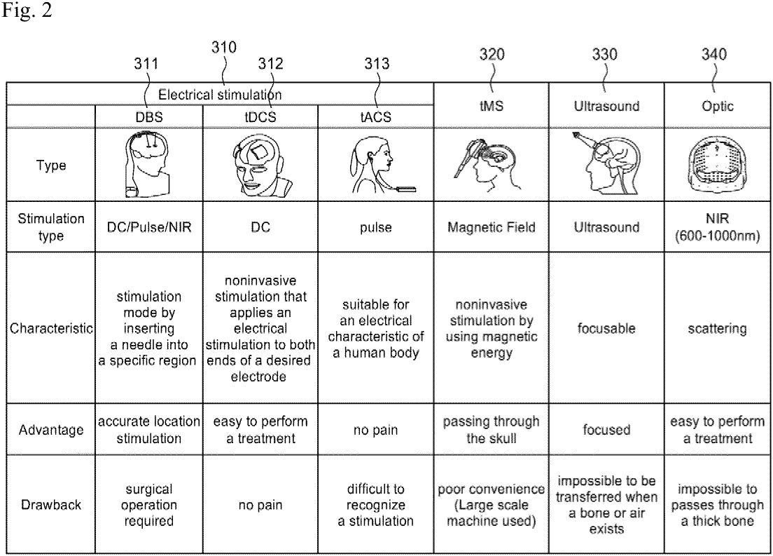

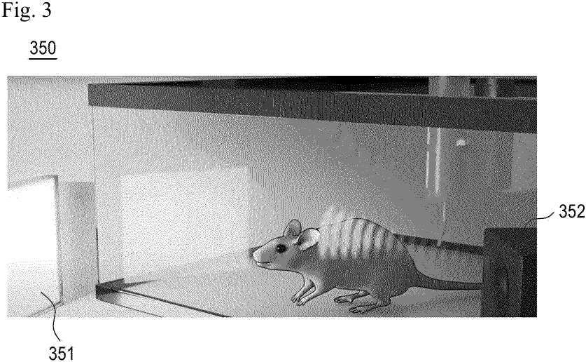

FIGS. 2 and 3 show a view explaining stimulation modes relating to the present disclosure.

Referring to FIG. 2, a DBS 311 refers to a stimulation mode by inserting a needle into a specific region, allowing an accurate location stimulation. However, this has a drawback that needs a surgical operation.

A tDCS 312 refers a noninvasive stimulation mode that applies an electrical stimulation to both ends of a desired electrode. This is easy to perform a treatment but has a risk of a burn around the electrode.

A tADC 313 refers to a stimulation mode that applies a pulse suitable for an electrical characteristic of a human body. This causes no pain but has a drawback that a stimulation is hardly recognizable.

A tMS 320 refers to a noninvasive stimulation mode using magnetic energy, allowing a stimulation passing through the skull but this lacks convenience (large scale machine used).

Ultrasound 330 is focusable but has a drawback that this is not transferable when a bone or air exists.

Light 340 is easy to perform a treatment but has a drawback that his hardly passes through a thick bone.

Meanwhile, a short range communication and a long range communication may be applicable between the server 200 and the brain stimulation device 100.

The wireless communication technology useable herein includes WLAN(Wireless LAN)(Wi-Fi), Wibro(Wireless broadband), Wimax(World Interoperability for Microwave Access), HSDPA(High Speed Downlink Packet Access), etc.

Further, the short range communication technology may include Bluetooth, RFID(Radio Frequency Identification), IrDA(infrared Data Association), UWB(Ultra-Wideband), ZigBee, etc.

Even not illustrated, a smart phone, a PC, etc. are applicable instead of the server 200.

Further, FIG. 3 shows a view for explaining a sensory stimulation mode relating to the present disclosure.

Referring to FIG. 3, shown is an example that a mouse is provided with a sensory stimulation through light and an auditory sense.

An optical stimulation which the mouse receives may be provided differently depending on period, pattern, intensity, etc.

For example, a full-field irradiation is performed by using a white stroboscope flash having 100 thousands of luminous intensity with fixing a stroboscope valve 20 cm before and after the eye of the rat. The stimulation is performed by applying a stimulation in the order from a low frequency stimulation to a high frequency stimulation for 10 seconds, observing a brain wave or an examinee for the next 10 seconds, and then proceeding with a next step.

An auditory stimulation part 352 refers to a therapy that stimulates a damaged brain with a sound as a medium and activates a brain function, recovering the brain function.

A neurological auditory stimulation therapy is subject to that activities recognizing a sound and generating stimulation affect the human brain and a behavior function. This is used for the purpose of rehabilitation of cognitive, motional and communicational functions damaged from stroke, brain damage resulting from an external injury, Parkinson’s disease, Huntington’s disease, Alzheimer’s disease, autism, other neurological diseases, etc.

### Alzheimer’s Disease Therapy Through Gamma Wave of Oscillation (γ-Oscillation) and Mechanism Thereof

The present disclosure intends to suggest to a new Alzheimer’s disease therapy through gamma wave of oscillation (y-oscillation) regulatory mechanism of a brain wave.

Imbalance in the neural network activity of neurons in the brain appears as a common pathological phenomenon of various brain diseases.

It is well known from recent various nonclinical and clinical trials, that, in the brain of a normal person, a balanced brain network activity between an excitatory neuron and an inhibitory neuron normally works and a brain activity of gamma wave of oscillation (of 25 to 100 Hz broadly, of 30 to 50 Hz restrictively) closely relates to a brain cognitive function and a memory function.

In various degenerative and psychiatric brain diseases (Alzheimer’s disease, Parkinson’s disease, epilepsy and schizophrenia), abnormal gamma wave of oscillation occurs by imbalance of transmission between GABAergic and glutamatergic agents secreted from neurons by activities of the excitatory neuron and the inhibitory neuron.

Thus, the present disclosure intends to suggest an apparatus and a method for treating Alzheimer’s disease through activation of gamma wave of oscillation and additionally a new approach for real-time monitoring.

A gamma entrainment therapy suggested by the present disclosure allows removing amyloid-β and tau proteins and increasing blood flow and cognitive ability in an AD animal model and a patient. Thus, this may become a new AD therapy.

Further, the present disclosure intends to suggest a method for prognosing a therapeutic effect of Alzheimer’s disease by monitoring a treatment process using gamma wave of oscillation as EEG (electroencephalography) and for visualizing a change in the an amyloid-β protein into PET.

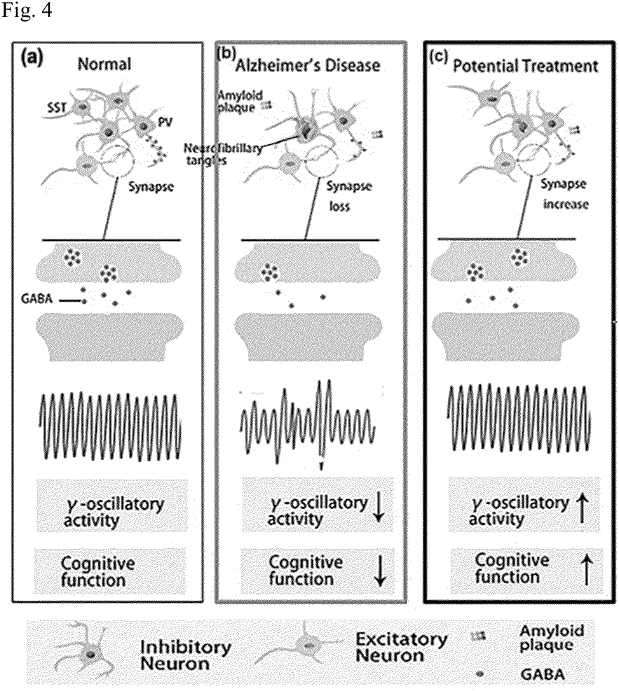

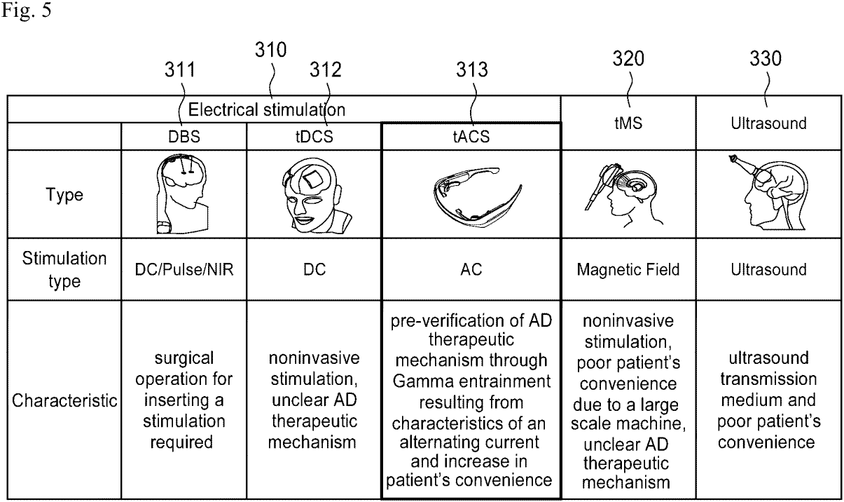

FIG. 4 shows a view for explaining a mechanism of an Alzheimer’s disease therapy through entrainment of gamma wave of oscillation according to the present disclosure.

Referring to section (a) of FIG. 4, a normal brain is shown.

Wherein, inhibitory and excitatory neurons are shown.

Transmission between GABAergic and glutamatergic agents is made with a communication through a synapse between the inhibitory and excitatory neurons.

In the normal brain, a chemical balance occurs while communicating. Agents secreted as shown in the section (a) of FIG. 4 generate gamma wave of oscillation of 20 to 100 Hz according to the balance, measured gamma wave of oscillation (y-oscillation) appearing normally and a cognitive function working normally.

However, as shown in section (b) of FIG. 4, in the case of a patient with Alzheimer’s disease, measured gamma wave of oscillation (y-oscillation) appears abnormally due to imbalance between GABAergic and glutamatergic agents secreted form inhibitory and excitatory neurons. Then, the GABAergic and glutamatergic agents decrease and amyloid-β and tau proteins are accumulated, thus lowing gamma wave of oscillation (y-oscillation) more, and falling a cognitive function definitely as compared to that of a normal person.

Through a gamma entrainment therapy (GET), a periodical spiking response may be induced in 5% or more of a recording region within a cortex area selected from hippocampus (HPC) and medial prefrontal cortex (mPFC).

The gamma oscillation is induced in a fast-spiking parvalbumin (FS-PV) interneuron, “fast-spiking (FS)” referring to an ability of a neuron capable of long-term release in a state that there is almost never spike frequency adaptation nor attenuation in a spike height.

In the end, a local field potential (LFP) is induced at about 40 Hz, allowing stabilizing a microglial activity and removing amyloid-β/tau.

Further, regarding the real-time monitoring & stimulation change, either an absolute threshold or the minimum amount of a sense needed for leading a response from a receptor may be changed, based on a stimulation type and an object.

Further, the monitoring is allowable based on a detection device that detects whether gamma oscillation is induced or not, an individual sensitivity, a cognitive function, a physiological or chemical change, a stress, safety and a stimulation and/or an individual, providing feedbacks therefor.

Therefore, as shown in section (c) of FIG. 4, the present disclosure suggests a therapy/mechanism that induces entrainment of gamma wave of oscillation (y-oscillation) synchronized in a patient with Alzheimer’s disease, etc., normalizing abnormal gamma wave of oscillation (y-oscillation), thereby improving a cognitive function.

### tACS Most Suitable for Entrainment of Gamma Oscillation, Adopted in The Present Disclosure

FIG. 5 show a view for explaining an advantageous effect of a transcranial alternating current stimulation (tACS) that is the most suitable for entrainment of gamma wave oscillation, in accordance with the present disclosure.

In a case of the DBS 311 among various stimulation modes described through FIGS. 2 and 3, this has a drawback that needs a surgical operation for inserting an electrical stimulation.

Further, the tDCS 312 is such a direct current mode, hardly inducing gamma wave of oscillation (y-oscillation) based on an alternating current mode.

Further, the tMS 320 is, as a large scale device, poor in patient’s convenience and has a difficulty in applying the mechanism described in FIG. 4 thereto.

Further, the ultrasound 330 essentially needs an ultrasound transmission medium and has, as a large scale device, a problem falling patient’s convenience.

Therefore, the present disclosure intends to suggest a solution for existing problems through a personalized transcranial alternating current stimulation (tACS) apparatus, and a new way of therapy.

A tACS 313 device uses an alternating current suitable for gamma entrainment therapy and has an advantage that a definite therapeutic mechanism for AD using this has been already verified as compared to other electrical stimulation modes (DBS, tDCS), or tMS and ultrasound modes.

Further, it is allowable to develop the tACS 313 as a compact, wearable device as compared to other devices, and needs neither surgical operation that is needed in the DBS nor a medium that is needed in the ultrasound mode, providing a reasonable price and patient’s most convenience. Further, safety verification thereof is secured according to the standard of the FDA and KFDA (2mA used).

### Apparatus for Sensing a tACS and a Brain Wave

According to the present disclosure, it is capable of providing a customized stimulation through AI-based EEG measurement.

That is, an apparatus for sensing a tACS and a brain wave is configured to determine a stimulation pattern through EEG measurement, to perform real-time monitoring and to provide a user with an optimal stimulation.

User’s EEG is measured, a current state of the brain is determined based on a measured EEG, and optimal intensity, period, pattern, etc. of a stimulation may be determined based on accumulated data.

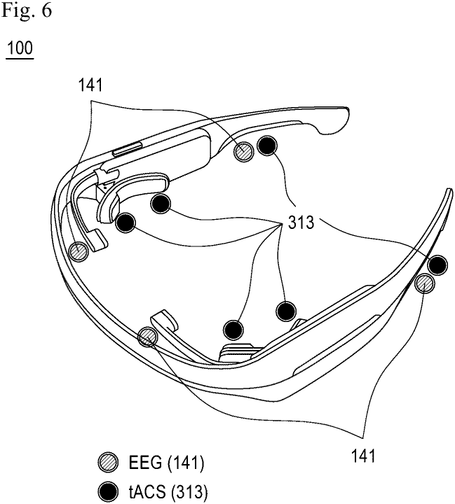

FIG. 6 shows a view for explaining an apparatus that senses a tACS and a brain wave.

Referring to FIG. 6, the brain wave measurement part 141 and the tACS part 313 are placed in a plural number.

Further, for a communication with the server 200, a current state of the brain is determined based on a measured EEG and optimal intensity, period, pattern, etc. for improving a determined brain state may be settled based on the accumulated data.

Hereinafter, referring to the accompanying drawing, in detailed described are a combined stimulation mode through the tACS part 313 and a closed loop neuro-feedback mode based on real-time measurement of a brain wave.

### EEG Signal-Based Closed Loop Neuro-Feedback tACS Mode

Conventionally, it is impossible to induce entrainment of gamma wave of oscillation (y-oscillation) synchronized according to a tACS suggested by the present disclosure and sense this in real time.

That is, for GET (gamma entrainment therapy), a technique for monitoring a brain signal in real-time is necessary. However, gamma oscillation falls in a domain of 25 to 100 HZ and a target frequency thereof is 40 Hz in general. Further, since excitation is achieved at 40 Hz, frequencies to be measured through an excitation frequency and EEG are in the same domain and thus real-time monitoring is impossible technically.

For example, when applying a 40 Hz stimulation for entrainment of gamma wave of oscillation, since a sensing through EEG targets a signal in a 40 Hz band, there was a big problem in determining which sort of a measured signal falls in - a noise came from a stimulation, or a target EEG signal. Thus, a method suggested by the present disclosure could not be implemented in the prior art.

Therefore, in the present disclosure, the tACS mode for acquiring an EEG signal in real time uses a combined mode of a burst frequency and a pulse frequency (PRF), allowing separating an EEG frequency from a stimulation frequency band.

That is, a frequency applied to a combined stimulation follows a pulse repetition frequency (PRF) in a 30 Hz to 50 Hz band to induce entrainment of oscillation synchronized in a plurality of brain areas of an object.

Further, the combined stimulation is a signal having a magnitude enough to induce a membrane action potential and brain oscillation in the plurality of brain areas of the object, this signal having a form of a burst signal that is ON/OFF according to the PRF rather than continuously applied.

Further, an ON/OFF signal according to the PRF is applied as a stimulation according to a burst frequency, wherein the burst frequency is set higher than the pulse repetition frequency (PRF).

For example, a RPF to be ON/OFF uses a low frequency of 40 Hz (for the purpose of inducing γ-oscillation) and a burst frequency of an ON signal is 10 kHz, allowing using a high frequency (for the purpose of inducting a membrane action potential and brain oscillation).

Wherein, a signal by the combined stimulation is of a high frequency band (10 kHz), and thus this is separated through a low pass filter in order to measure a frequency of a target EEG signal (40 HZ). Further, a magnitude of an electrical signal generated by the PRF is approximately 100 times lower than that of the target EEG signal, thus acquiring a brain wave signal induced by the combined stimulation in real time without interference.

That is, when applying a 40 Hz electrical stimulation to receive a 40 Hz EEG in real time, real-time monitoring is not possible due to interference of the electrical signal and the EEG signal.

Therefore, in the present disclosure, a high frequency of burst electrical stimulation is transferred through a 40 Hz RPF, thus inducing gamma entrainment.

At this time, since the electrical signal by the burst stimulation is sorted as a high frequency band, allowing signal separation, it is capable of sensing the 40 Hz EEG signal in real time.

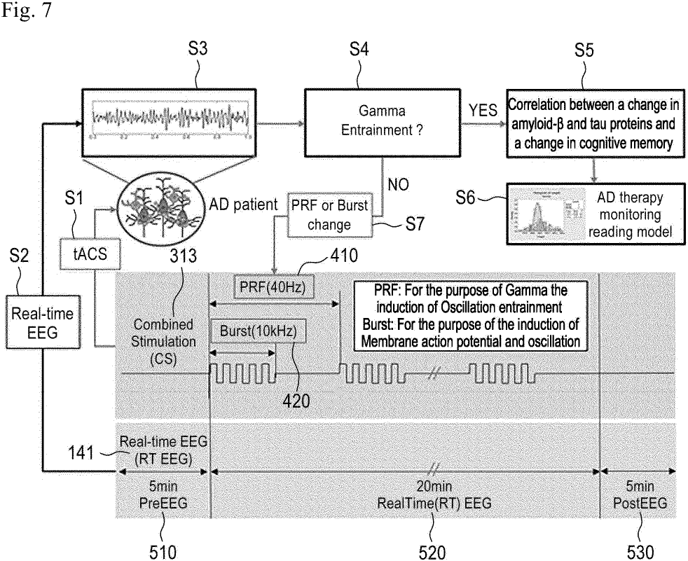

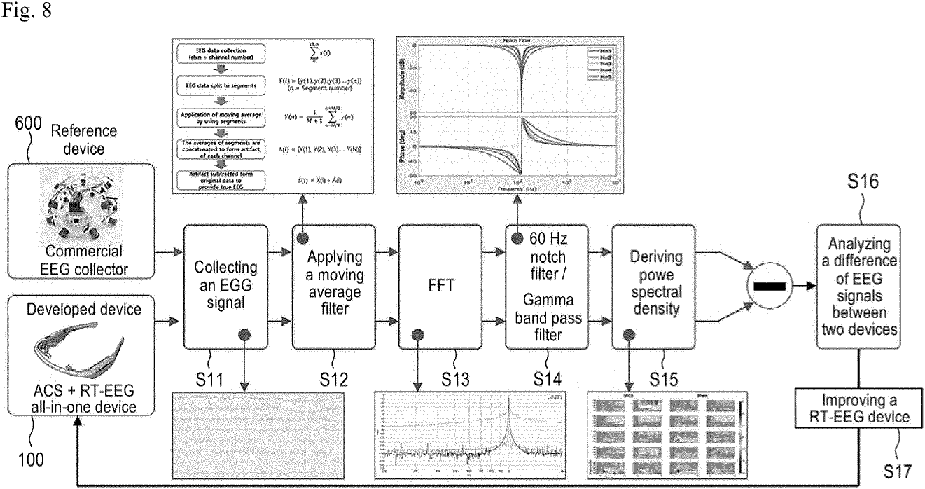

Referring to FIG. 7, more detailed contents are described.

FIG. 7 shows a view for explaining an EEG-based closed loop neuro-feedback tACS mode suggested in the present disclosure.

Referring to FIG. 7, to separate an EEG frequency from a stimulation frequency band, the tACS 313 mode may be performed by using a combined stimulation mode of a burst frequency and a pulse repetition frequency (PRF).

The combined stimulation is a burst signal that is ON/OFF according to a pulse repetition frequency (PRF) 410 to which a signal to be applied is predetermined. An ON signal according to the PRF 410 is applied as a stimulation according to a burst frequency 420.

As described above, the PRF 410 is applied to induce entrainment of oscillation synchronized in a plurality of brain areas of an object, and the burst frequency 420 is applied to induce a membrane action potential and brain oscillation in the plurality of the brain areas of the object.

Wherein the burst frequency 420 is 10 kHz, having a higher value than the 40 Hz PRF 410.

The burst frequency 420 makes a neuron move through the membrane action potential and induces the brain oscillation, allowing a communication.

The burst frequency 420 is stimulated with a high frequency, allowing easily inducing a membrane activity and rapidly inducing oscillation. Furthermore, the burst frequency 420 is set much higher than a 30 Hz to 50 Hz band to differentiate a brain signal induced by the pulse repetition frequency (PRF) 410 of the combined stimulation generated in the brain according to the PRF 410 from an applied combined stimulation.

Further, the pulse repetition frequency (PRF) 410 is applied to induce entrainment of the synchronized gamma oscillation described above and induces an activity stimulated by the burst signal 420 to be synchronized according to the gamma oscillation and entrained.

Particularly, the pulse repetition frequency (PRF) 410 may be a transfer mode of a stimulation having a frequency of 30 Hz to 50 Hz to an object to entrain gamma oscillation synchronized in a plurality of brain areas of the object including prefrontal cortex (PFC) and hippocampus of the object.

Once a combined stimulation is applied to the brain, a neuronal activity is induced by the burst frequency 420, an irregular communication occurring, wherein a neuron of which activity was induced by a period of a gamma wave domain according to the PRF 410 is entrained and operated.

Therefore, in the present disclosure, brain wave entrainment is induced through a high frequency of burst stimulation, and gamma-entrainment is induced through the 40 Hz RFP.

Furthermore, the burst frequency 420 of a combined stimulation to be applied is set much higher than the 30 Hz to 50 Hz band. Thus a brain signal induced the PRF 410 of the combined stimulation is differentiated from a combined stimulation to be applied, allowing differentiating and acquiring only a target brain wave as ignoring a sensed combined stimulation.

In the past, a PRF had to be applied at 40 Hz and 40 Hz had to be sensed with EEG, and thus real-time measurement was not available. Like the present disclosure, when applying a burst signal that is ON/OFF according to the burst frequency 420, as a combined stimulation according to the PRF 410 for inducing entrainment of oscillation synchronized in a brain area, as shown in an example of FIG. 8, there is a difference of 40 dB or more (100 times or more when mathematically converted), allowing differentiating a signal according to the combined stimulation from a brain wave signal induced by the PRF 410 of the combined stimulation and also allowing acquiring only a targeting brain wave signal.

The sensing part 140 is a brain wave measurement part that measures a brain wave of the brain induced corresponding to the stimulation. The control part 180 determines whether a response is derived or not by using a determination of whether a measured brain wave corresponds to an output and a wave form of a entrained brain wave that appears only when entraining the γ-oscillation or not.

The control part 180 calculates an average of power spectrum values of respective frequency bands and a standard deviation thereof, and a ratio of each average value according to at least one combination of gamma (γ)/ alpha (α)/ beta (β)/ delta (δ)/ theta (θ) brain waves from the measured brain wave, thus extracting a property of the measured brain wave.

An EEG sensor 141 according to the present disclosure measures a signal in real time, for example, sensing a signal that is not used as a pre EEG signal 510 for 5 minutes and then sensing an RT EEG signal 520 that is used for real-time analysis for the next 20 minutes.

Further, following measuring the RT EEG signal 520 for 20 minutes, this is processed with a signal that is not used as a post EEG signal 530 for 5 minutes once more.

As described above, the purpose of the real-time EEG measurement in the present disclosure is to confirms whether synchronized gamma oscillation is actually entrained in a user through the apparatus provided by the present disclosure or not.

Generally, as a combined stimulation is applied according to the PRF 410 of 40 Hz, the user (patient) may be entrained to synchronized gamma oscillation.

However, in a case of a patient with Alzheimer’s disease (AD), as the patient’s cognitive function is poor, entrainment of synchronized gamma oscillation may be achieved at a frequency lower than 40 Hz.

Therefore, in the present disclosure, as transferring the burst frequency 420, the PRF 410 and a combined stimulation resulting from correction of at least one of a wave form and a period of a stimulation to an object, entrainment of synchronized gamma oscillation may be actually induced.

Typically, in a case that the PRF 410 is applied at 40 Hz at first but the entrainment is not induced but the entrainment is not induced, the entrainment may be induced by applying signals of 38 hz, 36 Hz, 34 Hz, 32 Hz one by one with lowering each frequency thereof.

Through continuous use of the present apparatus, patient’s brain function is improved, allowing inducing entrainment at a 40 Hz band falling in a normal range in a patient in who entrainment is generally induced at a 32 Hz band.

On the contrary, likely there is a patient in whom entrainment of gamma oscillation is induced at a frequency more than 40 Hz, and thus it is allowable to apply a stimulation by correcting a combined stimulation in a way of increasing a frequency.

Referring to FIG. 7, through the tACS part 313, a combined stimulation is applied according to the PRF 410 for inducing entrainment of oscillation synchronized in the brain area in a burst signal that is ON/OFF according to the burst frequency 420 (S1).

Further, the EEG sensor 141 senses a brain signal according to the combined stimulation while proceeding with stimulation (S2, S3).

At this time, in a control part 180, the EEG sensor 141 sorts a signal by the combined stimulation among measured signals into a noise, allowing ignoring the signal.

Further, the control part 180 determines whether a response entraining gamma oscillation based on a brain wave signal induced by the PRF 410 of the combined stimulation among the measured signals is derived or nor.

When a response entraining γ-oscillation is not derived, the control part 180 transfers the burst frequency 420, the PRF 410 and a combined stimulation resulting for correction of at least one of a wave form and a period of a stimulation to the brain of the object, allowing actually inducing entrainment of gamma oscillation (S7).

The S1 to the S7 may be performed repeatedly until the gamma oscillation actually synchronized in the brain of a user is actually entrained.

When the synchronized γ-oscillation is actually entrained, the control part 180 analyzes correlation between a change in amyloid-β and tau proteins and a change in cognitive memory (S5), allowing providing AD therapy monitoring information through a display part 151.

In the end, the present disclosure increases an AD therapeutic effect by a neurofeedback mode through a determination of whether gamma entrainment is achieved or not using a technique for acquiring an EEG signal when applying a tACS in real time.

### aACS+EEG All-in-One Device

FIG. 8 shows one example of a diagrammatic view for development of a tACS+EEG all-in-one device, in accordance with the present disclosure.

Referring to FIG. 8, in order to verify EEG signal collection based on the tACS+EEG all-in-one device suggested by the present disclosure, a commercial EEG signal collector (OpenBCI) 600 may be used as a reference device together.

EEG signals are collected from each device (S11) and a moving average filter is applied on a time axis based on collected EEG signals (S12), followed by changing a frequency axis through FFT (Fast Fourier Transform) (S13). Then, a rated voltage is removed and a filter for observing only a gamma band is applied (S14). A difference between power spectrum densities (PDS) of respective signals is induced (S15) and a distortion degree of a tACS+EEG all-in-one device is relatively analyzed (S16). It, hereby is possible to improve an EEG performance on the device according to the present disclosure.

In the present disclosure, performance enhancement of the tACS+EEG all-in-one device is achieved through comparative analysis with the commercial EEG collector 600 to confirm gamma entrainment, allowing re-verification of an optimal stimulation parameter for tACS.

It, hereby is allowable to proceed with treatment of a user based on an ultimately selected stimulation parameter for gamma entrainment.

### Real-Time tACS-EEG Neurofeedback Algorithm

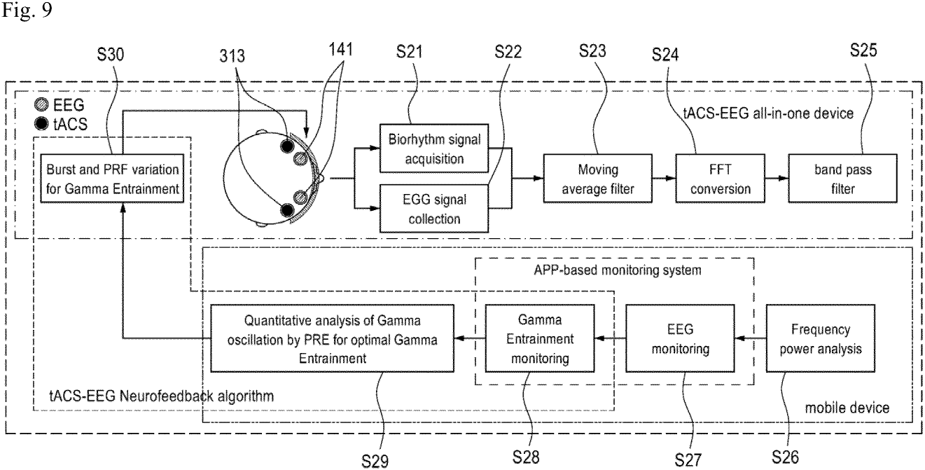

FIG. 9 shows one example of a block view of a real-time tACS-EEG Neurofeedback algorithm, in accordance with the present disclosure.

As described above, each patient has a different period of gamma band. Thus, it is critical to apply a tACS signal having a PRF period optimized for each patient in order for efficient gamma entrainment. Further, various frequency characteristics such as biorhythm signals, etc. that are different from each other depending on a patient are mixed with an EEG signal and thus a difficulty in analysis of EEG signals for ideal gamma entrainment occurs.

Therefore, as shown in FIG. 9, developed is a real-time Neurofeedback algorithm that allows a tACS+EEG all-in-one device to induce optimized gamma entrainment depending on a patient by itself (S28, S29, S30). The algorithm may be composed of an EEG gamma oscillation analysis algorithm that confirms a period of a gamma band of a patient in advance (S27 and S28) and allows applying a tACS signal having an optimal PRF, and of a moving average filter algorithm that confirms a biorhythm signal depending on a patient in advance and allows real-time removal thereof from collected EEG signals (S21 to S26).

### Derivation and Visualization of Correlation Between an Aβ PET Image and a Gamma Band

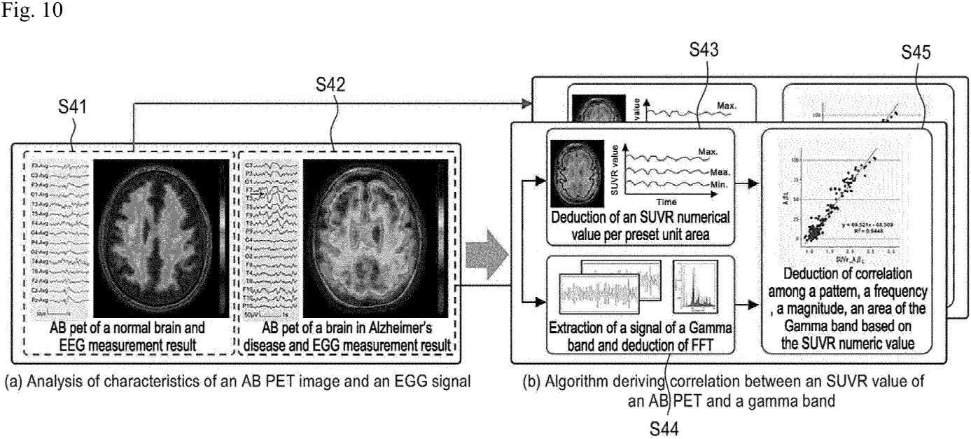

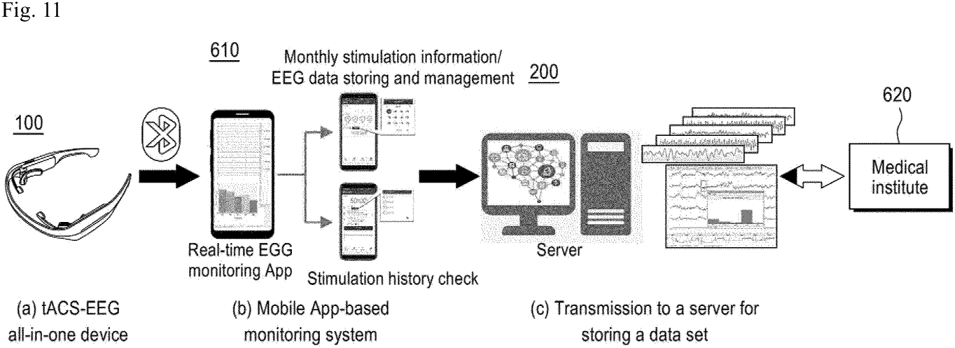

FIG. 10 shows one example of derivation and visualization of a correlation between an Aβ PET (Amyloid beta PET) image and a gamma band, in accordance with the present disclosure.

Section (a) of FIG. 10 is one example for the analysis of an Aβ PET image and an EEG signal characteristic, and section (b) is one example for an algorithm deriving correlation between an SUVR value of the Aβ PET image and the gamma band.

The Aβ PET image shows a characteristic that the more severe a degree of Alzheimer’s disease is, the more scale numerical value of an SUVR parameter increases. Thus, correlation of the tACS+EEG all-in-one device suggested by the present disclosure with gamma entrainment efficiency is derived, allowing monitoring the AD treatment process.

For this, in the present disclosure, an SUVR numerical value per preset unit area of an Aβ PET image before and after stimulation is derived using a tACS+EEG device 100. The numerical value may be applied to an algorithm that computes correlation thereof with parameters such as collected time axis pattern, frequency axis area, etc. of the gamma band.

In the end, through building a database of the correlation between the SUVR parameter of the Aβ PET and the gamma band, it is possible to provide an algorithm that predicts a therapeutic effect based on artificial intelligence.

### Therapeutic System Through App-Based EEG Monitoring and Cooperation with an External Medical Institute

It is important to early diagnose dementia and to confirm treatment therefor by applying the tACS+EEG all-in-one device 100 suggested by the present disclosure as well as continuously storing a pattern and characteristic data of an EEG signal collected in real time through the device 100 .

FIG. 11 shows one example of a therapeutic system through App-based EEG monitoring and cooperation with an external medical institute, in accordance with the present disclosure.

Section (a) of FIG. 11 shows the tACS+EEG all-in-one device 100, section (b) showing an App-based EGG monitoring system 610, section (c) showing a server 200 that receives data transmitted to a mobile, and section (d0 showing a predetermined medical institute 620.

Referring to FIG. 11, the App-based EEG monitoring system 610 is applied which is capable of data linkage with the mobile through a wireless communication with the tACS+EEG all-in-one device 100. The data transmitted to the mobile is transmitted to the server 200. The server 200 transfers this data to the medical institute 620 and receives a medical personnel’s determination. A dataset for a digital biomarker based thereon is secured and then used.

According to the present disclosure, it is allowable that a control part 180 of the device 100 performs determination of whether gamma entrainment is induced or not and also receives the medical personnel’s determination through the server 200.

Furthermore, it may be also determine whether gamma entrainment is induced or not by combining determinations from the control part 180 of the device 100 and the server 200.

### Effects of Entraining Gamma Oscillation Synchronized in the Brain Area

Dementia including Alzheimer’s disease (AD) is a fatal brain disease characterized by deterioration in the brain and a cognitive function (Canter).

Various factors join in AD pathogenesis including amyloid-β deposition, hyperphosphorylated tau accumulation, microgliacyte- and astrocyte-mediated inflammation, and losses of neuron and synapse.

According to a study result, it was observed that a change in the neural activity affects AD pathology such as amyloid-β and tau accumulation in several mouse models.

As taking this observation into account, various kinds of approaches are used to examine whether manipulation of neuronal oscillation is effective on improvement of the AD pathology or not.

Particularly, it was revealed that oscillation at a gamma frequency band (of about 30 to 90 Hz) was reduced in several kinds of mouse models including hAPP-J20, ApoE4 and 5XFAD, as well as in a human patient with AD.

Some of recent studies targeted gamma oscillation and results thereof show that this may be a promising strategy for alleviating the AD pathology.

Firstly, the increase in the gamma oscillation through expression of a voltage-gated sodium channel subunit Nav1.1 existing in a parvalbumin-positive (PV+) interneuron, or by neural transplantation of an Nav1.1 overexpressed interneuron precursor cell, alleviated a gamma lack in a hAPP-J20 mouse and declined both an epileptic activity and a cognitive performance.

Further, it is revealed that an optogenetic activity of the PV+ interneuron at 40 Hz, which appeared by inducing a strong gamma frequency oscillation, reduces an amyloid load and improves morphological deformation of microglia.

Such a noninvasive approach using a 40 Hz stimulation reduces the amyloid load and is similarly effective on the deformation of the microglia.

Furthermore, it is revealed that shown are decreases in the level of amyloids (in soluble and insoluble forms of Aβ1-40 and Aβ1-42) as well as plaque pathology.

Further, various cell types (neuron and microglia included) that decrease generation of Aβ and increase removal thereof respectively in a 5XAFD mouse model were affected by inducing the strong gamma frequency oscillation.

Therefore, as inducing gamma oscillation synchronized in the brain through a brain stimulation, the amyloid load is declined and the morphological deformation in a part of the brain area appears.

An overall fundamental technology of the present disclosure induces entrainment of gamma oscillation in the brain of an object through stimulation, and expands a gamma interference to brain areas (for example, hippocampus, somatic sensory cortex and prefrontal cortex) as well as reinforcing a low gamma interference over these brain areas.

That is, the present disclosure may stimulate the brain of the object by using at least one of sensory stimulations noninvasively transferring at least one of an electrical stimulation, an ultrasound stimulation, an optical stimulation, a vision stimulation and an auditory stimulation.

The present disclosure may induce a local field potential (LFP) of 40 Hz in prefrontal cortex and hippocampus of the object based on entrainment of gamma oscillation.

Further, the present disclosure may regulate the neuronal activity between the brain areas of the object including prefrontal cortex and hippocampus through entrainment of gamma oscillation, providing an effect on neurodegeneration decreases.

Further, the present disclosure may decline amyloid plagues in the brain area of the object including prefrontal cortex and hippocampus and hyperphosphorylation of tau through entrainment of gamma oscillation.

Further, the present disclosure may reduce losses of neurons and synapses, brain atrophy, bulging of a ventricle in the brain and neuroinflammation, in brain areas of the object including PFC and hippocampus through entrainment of gamma oscillation.

Further, the present disclosure may reduce an immune response of at least a part of microglia, deforming the microglia morphologically and increasing proteolysis in the microglia, in brain areas of the object including, prefrontal cortex and hippocampus, through entrainment of gamma oscillation.

Further, the present disclosure may improve abnormally transformed genes and proteins relating to at least one of membrane trafficking, intracellular trafficking, a synapse function, neuroinflammation, an apoptosis process and a DNA damage, in brain areas of the object including PFC and hippocampus through entrainment of gamma oscillation.

In the end, the present disclosure may provide a user with a therapy relating to Alzheimer’s disease, brain tumor, depression, stroke, schizophrenia, epilepsy, sleep disorder, etc.

In the present disclosure, as inducing gamma oscillation synchronized in the brain, the amyloid load may be declined and the morphological deformation in a part of the brain area may appear, thereby improving a cognitive function in the end.

The induction of gamma oscillation, particularly in an object suffering from a neurological disease or disorder or age-related degeneration enables restoration of a gamma oscillation rhythm damaged from the disease or disorder or the age-related degeneration or from one relating thereto (regulation of microglial activity).

Further, the induction of gamma oscillation may reduce the generation of Aβ such as a soluble Aβ peptide, an insoluble Aβ peptide, homotypic Aβ-40 and Aβ-12, etc. and reinforces removal thereof.

Further, the induction of gamma oscillation may prevent Aβ from accumulated in the brain of the object.

Further, other effects resulting from the induction of gamma oscillation may improve abnormally transformed genes and proteins relating to at least one of neuronal activity regulation/neural degeneration decrease/neuroinflammation decrease, membrane trafficking, intracellular trafficking, a synapse function, neuroinflammation, an apoptosis process and a DNA damage, between several brain areas of the object.

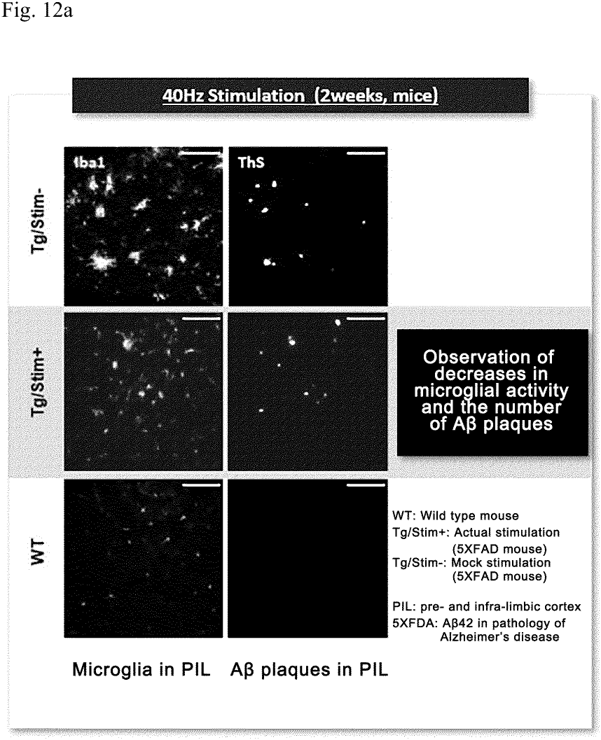

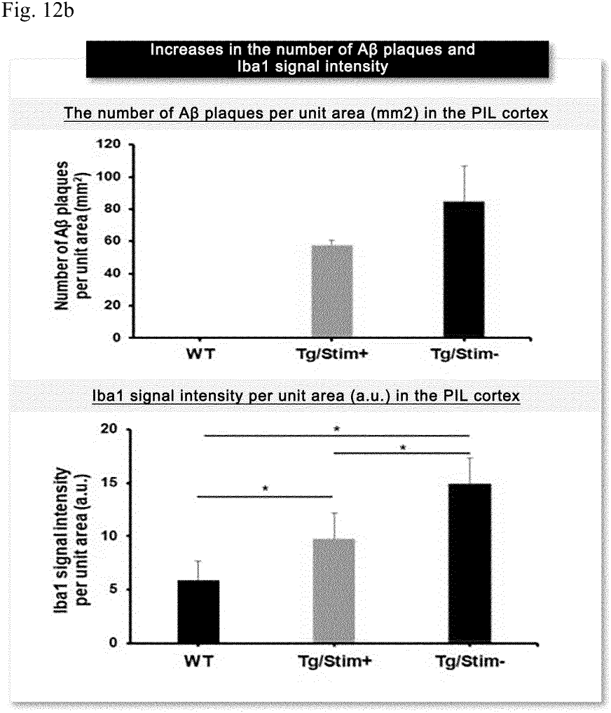

FIGS. 12A and 12B show test results for explaining reduction of amyloid-β plaques and an immune response of a gliacyte in the brain area by inducing entrainment of gamma-oscillation in a plurality of brain area of an object.

A microglia cell or a microgliacyte falls in microglia, referring to a neuron in charge of an immune function.

Brain health may be maintained by an appropriate level of exercise. However, in a case of the microglia acting as a leukocyte in the brain, the activity thereof is increased according to insufficient sleep and stress increases, thus inducing the accumulation of β-amyloids and taus according thereto.

FIG. 12A shows microglia in a pre- and infra-limbic cortex (PIL) and Aβ plaques in a PIL cortex, respectively.

FIG. 12A shows that a therapeutic stimulation of 40 Hz is irradiated to the brain, decreasing β amyloids and the microglial activity and improving a cognitive function, according thereto.

That is, decreases in the microglial activity and the number of Aβ plaques were observed, and 3 objects of each stage are as follows.
WT: Wild type mouse
Tg/Stim+: Actual stimulation (5XFAD mouse)
Tg/Stim-: Mock stimulation (5XFAD mouse)
PIL: pre- and infra-limbic cortex
5XFDA: Aβ42 in pathology of Alzheimer’s disease

Referring to FIG. 12A, regarding a mouse with Alzheimer’s disease, when inducing gamma oscillation, it is clearly seen that the microglial activity is decreased and the number of Aβ plaques is reduced.

Further, referring to FIG. 12 B, shown are the number of Aβ plaques per unit area (mm²) in the PIL cortex and an Iba1 signal intensity per unit area (a.u.) in the PIL cortex.

In FIG. 12B, it is seen that increases in the number of Aβ plaques at 40 Hz and the Iba1 signal intensity.

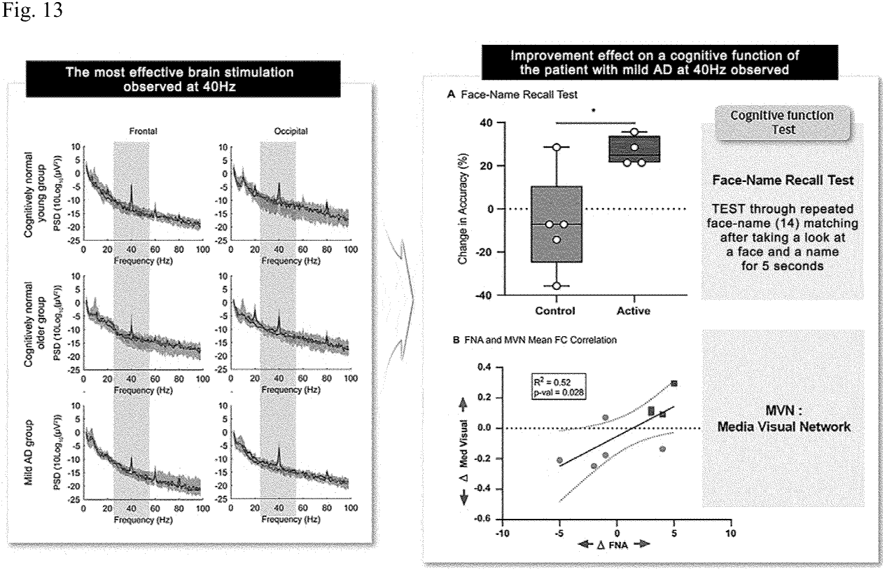

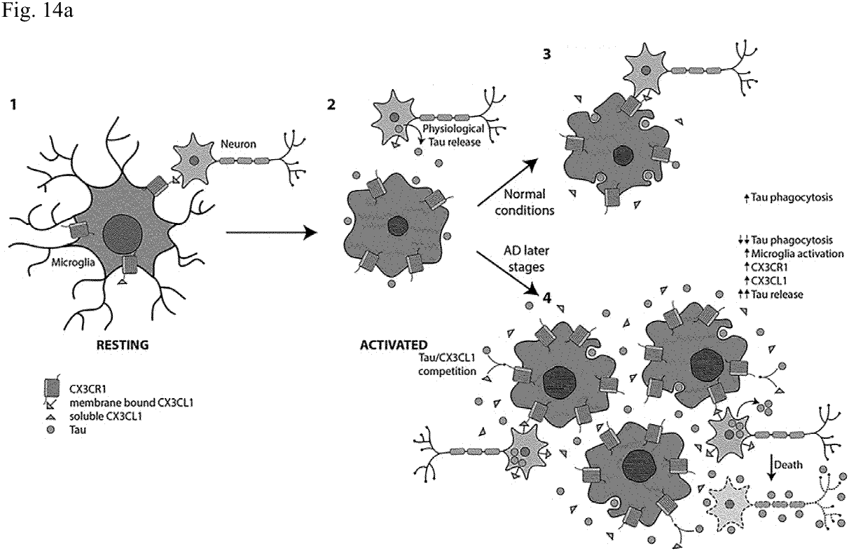

Meanwhile, FIG. 13 shows a view for explaining that a cognitive function of a patient with Alzheimer’s disease is improved by entraining gamma oscillation synchronized in a plurality of brain areas of an object.

Referring to FIG. 13, a randomized, placebo-controlled trial is performed targeting a patient with mild AD in order to confirm improvement in a memory performance and a cognitive function of the patient with mild AD according to a stimulation of 40 Hz.

Particularly, a 40 Hz sensory stimulation is applied to a control group once a day for 4 months. Then, measured are safety, compliance, a structure and functions of the brain, sleep and a change in a cognitive function. Ultimately, observed are high drug tolerance and compliance/ Efficacy & Safety at the stimulation of 40 Hz.

That is, induction of 40 Hz entrainment is stable as homecare and shows improvement effects on structural, functional degeneration relating to a cognitive function and AD.

Further, referring to FIG. 13, regarding a cognitive function test, a Face-name Recall Test is performed through repeated face-name matching after taking a look at a face and a name for 5 seconds and a result for proceeding with MVN (Media Visual Network) is shown, wherein an improvement effect on the cognitive function of the patient of mild AD is clearly observed at the induction of 40 Hz entrainment.

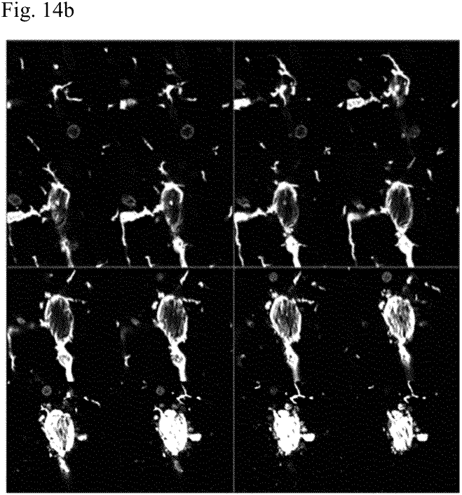

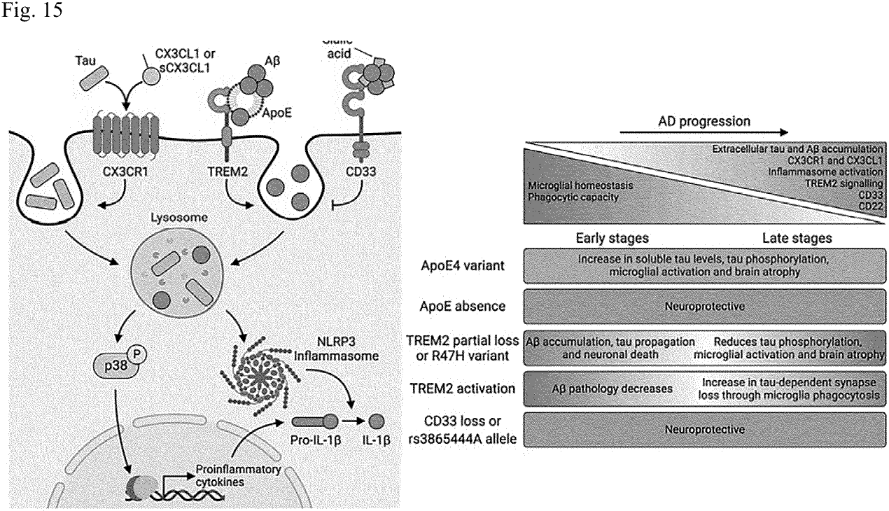

FIGS. 14A and 14B show views explaining a relation between a microglial activity and tau tangles, in accordance with the present disclosure, and FIG. 15 shows a view for explaining improvement of tau tangles by regulating a microglial activity through gamma-oscillation entrainment therapy.

Referring to FIGS. 14Aand 14B, shown are a process that tau tangle become worse, thus getting damaged as a microglial activity increases, and a test result.

Further, referring to FIG. 15, shown is a process that hyperphosphorylated tau aggregation and neurofibrillary tangles are reduced by alleviating the microglial activity.

Therefore, the present disclosure stimulates the brain of an object through a tACS and induces a local field potential (LFP) of 40 Hz is in hippocampus and prefrontal cortex of the object, based on entrainment of gamma oscillation through stimulation, thereby providing following effects to a user.
Regulating a neuronal activity, reducing neural degeneration
Decreasing amyloid plaques and tau hyperphosphorylation
Reducing losses of neurons and synapse, brain atrophy, bulging of a ventricle in the brain and neuroinflammation
Reducing an immune response of microglia, deforming microglia morphologically, increasing proteolysis
Improving abnormally transformed genes and proteins relating to at least one of membrane trafficking, intracellular trafficking, a synapse function, neuroinflammation, an apoptosis process and a DNA damage, in brain areas of the object including PFC and hippocampus

In the present disclosure, an anti-dementia drug is provided through microglia stabilization + amyloid/tau removal by applying an optimal stimulation pattern, based on the aforementioned AI-based EEG analysis.

Particularly, an optimal dose (intensity, frequency, period, pattern, etc.) may be applied as an optimal treatment option, based human-targeting neuromodulation stimulation data.

### Advantageous Effects According to the Present Disclosure

According to one embodiment of the present disclosure, it is capable of providing an apparatus and a method for stimulating the brain by entraining oscillation synchronized in a plurality of brain areas of an object.

Particularly, the present disclosure is capable of providing an apparatus and a method for deriving and applying an optimal stimulation by: stimulating the brain according to a typical stimulation mode determined corresponding to a condition of an object among a plurality of typical stimulation modes; and determining whether a response to entrainment of gamma oscillation synchronized in a plurality of brain areas of the object based on a measured brain response is derived or not.

The present disclosure is capable of transferring a stimulation having a frequency of 30 Hz to 50 Hz to an object for entraining gamma oscillation synchronized in a plurality of brain areas of the object including prefrontal cortex (PFC) and hippocampus.

The present disclosure is capable of providing a customized tACS (transcranial Alternating Current Stimulation) apparatus and a method for entraining gamma oscillation synchronized in the brain, based on real-time EEG signal monitoring.

The present disclosure is capable of providing a new AD therapy through a regulatory mechanism of γ-oscillation in a brain wave.

Imbalance in the neural network activity of neurons in the brain is a common pathological phenomenon of various brain diseases, and thus the present disclosure is capable of providing a new approach to an Alzheimer’s disease therapy and monitoring through activation of a gamma wave of oscillation (y-oscillation).

The present disclosure is capable of providing an optimal gamma entrainment parameter of a personalized tACS apparatus and a wearable tACS apparatus.

The present disclosure is capable of providing a “tACS+EEG all-in-one device” and an App-based EEG monitoring system.

The present disclosure is capable of providing verification of a gamma brain wave induction performance of a tACS apparatus targeting a normal aged person, and an optimal stimulation condition for brain wave entrainment different depending on an individual.

The present disclosure is capable of providing a personalized therapeutic algorithm for Alzheimer’s disease based on a neurofeedback algorithm.

The present disclosure is capable of providing methods for analyzing and representing brain stimulation and rehabilitation effects through building of a metaverse.

The present disclosure is capable of providing a system and a method for verifying and representing correlation between effects of Aβ PET (Amyloid beta PET) and cognitive function test-based Gamma entrainment.

The present disclosure is capable of inducing a local field potential (LFP) of 40 Hz in prefrontal cortex (PFC) and hippocampus of an object, based on entrainment of gamma oscillation through stimulation.

Further, the present disclosure is capable of providing effects that a neuronal activity is regulated between brain areas of the object including PFC and hippocampus and neural degeneration is declined, through gamma entrainment.

Further, the present disclosure is capable of reducing amyloid plaques and hyperphosphorylation of tau, in brain areas of the object including PFC and hippocampus, through entrainment of gamma oscillation.

Further, the present disclosure is capable of reducing losses of neurons and synapses, brain atrophy, bulging of a ventricle in the brain and neuroinflammation, in brain areas of the object including PFC and hippocampus through entrainment of gamma oscillation.

Further, the present disclosure is capable of reducing an immune response of at least a part of microglia, of deforming the microglia morphologically and of increasing proteolysis in the microglia, in brain areas of the object including, prefrontal cortex and hippocampus, through entrainment of gamma oscillation.

Further, the present disclosure is capable of improving abnormally transformed genes and proteins relating to at least one of membrane trafficking, intracellular trafficking, a synapse function, neuroinflammation, an apoptosis process and a DNA damage, in brain areas of the object including PFC and hippocampus through entrainment of gamma oscillation.

In the end, the present disclosure is capable of providing a user with the therapy relating to AD, Parkinson’s disease, stroke, epilepsy, schizophrenia, etc.

Meanwhile, advantageous effects to be obtained by the present disclosure are not limited to the aforementioned effects, and other not-mentioned advantageous effects may be clearly understood by the skilled person in the art to which the present disclosure pertains from the description below.

### Expansion of the Therapeutic Filed to Nervous System Diseases

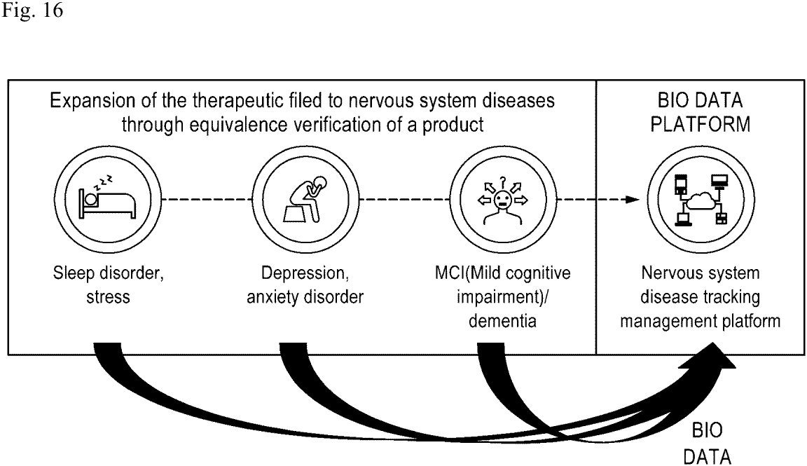

FIG. 16 shows a view for explaining expandability of the therapeutic field to nervous system diseases through equivalence verification of a product according to the present disclosure, in accordance with the present disclosure.

Referring to FIG. 16, a wearable tACS apparatus 100 is not limited to a stimulation of a gamma oscillation band, allowing generating a tACS signal for stimulating various brain waves of delta, theta, alpha, etc. through simple remodeling of a system.

Particularly, it is reported that a delta wave (0.5 to 3 Hz) and an alpha wave (8 to 12 Hz) are induced based on a deep sleep and psychological stability, respectively. Thus, it is allowable to use these as equivalent products relating to a sleep disorder, stress, depression, insomnia, etc. through frequency variation of the wearable tACS apparatus 100.

Meanwhile, embodiments of the present disclosure may be implemented in various ways. For example, the embodiments of the present disclosure may be implemented by hardware, firmware, software or a combination of the preceding or etc.

In a case of an implementation by the hardware, methods according to the embodiments of the present disclosure may be implemented by ASICs(Application Specific Integrated Circuits), DSPs(Digital Signal Processors), DSPDs(Digital Signal Processing Devices), PLDs(Programmable Logic Devices), FPGAs(Field Programmable Gate Arrays), a processor, controllers, micro-controllers, microprocessors, etc.

In a case of implementation by either firmware or software, methods according to the embodiments of the present disclosure may be implemented into forms of a module for performing the aforementioned functions or operations, a procedure or a mathematical function, etc. A software code is stored in a memory unit and may be driven by a processor. The memory unit is positioned inside or outside the processor and may receive and transfer data from and to the processor in various known ways.

Detailed descriptions of the preferred exemplary embodiments of the present disclosure disclosed as described above are provided so as for the skilled person in the art to implement and execute the present disclosure. The present disclosure has been described with reference to the preferred exemplary embodiments, but the skilled person in the art will understand that the present disclosure can be variously modified and changed without departing from the scope of the present disclosure. For example, the skilled person in the art may use the respective components disclosed in the exemplary embodiments by combining the respective components with each other. Therefore, the present disclosure is not limited to the exemplary embodiments described herein, but intends to grant the widest range which is coherent with the principles and new features disclosed herein.

The present disclosure may be embodied in other specific forms without departing from the spirit and essential characteristics of the present disclosure. Accordingly, the aforementioned detailed description should not be construed as restrictive in all terms and should be exemplarily considered. The scope of the present disclosure should be determined by rational construing of the appended claims and all modifications within an equivalent scope of the present disclosure are included in the scope of the present disclosure. The present disclosure is not limited to the exemplary embodiments described herein, but intends to grant the widest range which is coherent with the principles and new features presented herein. Further, the claims that are not expressly cited in the claims are combined to form an exemplary embodiment or be included in a new claim by an amendment after the application.

What is claimed is:

1. An apparatus comprising:
a stimulation part that stimulates a brain of an object;
a sensing part that measures a response of the brain to a stimulation; and
a control part that determines whether a first response entraining oscillation synchronized in a plurality of brain areas of the object is derived or not, based on a measured brain response, wherein
when the first response is not derived, the control part controls to stimulate the brain of the object according to a corrected stimulation mode.

2. The apparatus of claim 1, wherein
the stimulation part is an electrical stimulation part that transfers a transcranial alternating current stimulation (tACS),
the tACS is a first combined stimulation in which a signal is repeatedly ON/ OFF according to a predetermined first frequency and an ON signal according to the first frequency is applied as a stimulation according to a predetermined second frequency,
the control part processes a first signal by the first combine stimulation among signals measured by the sensing part as a noise, and determines whether the first response is derived or not, based on a second signal except the first combined stimulation among the signals measured the sensing part,
when the first response is not derived according to the first combined stimulation, the control part controls the electrical stimulation part to transfer, to the brain of the object, a second combined stimulation resulting from correction of at least one of the first frequency, the second frequency, and an output, a waveform and a period of a stimulation according to the second frequency.

3. The apparatus of claim 2, wherein
the first frequency is applied to induce entrainment of oscillation synchronized in the plurality of brain areas of the object,
the second frequency is applied to induce a membrane action potential and brain oscillation in the polarity of brain areas of the object, and
the second frequency has a higher value that the first frequency.

4. The apparatus of claim 3, wherein
the stimulation part stimulates the brain of the object according to a stimulation mode determined corresponding to a condition of the object,
the condition of the object denotes a disease relating to the object and a progressive degree of the disease, and
the disease includes Alzheimer’s disease, Parkinson’s disease, stroke, epilepsy and schizophrenia.

5. The apparatus of claim 4, wherein
the first response is about entrainment of gamma oscillation synchronized in the plurality of brain areas of the object,
the first frequency is a frequency of 30 Hz to 50 Hz for entraining the gamma oscillation synchronized in the plurality of brain areas of the object including prefrontal cortex (PFC) and hippocampus.

6. The apparatus of claim 5, wherein
an interference signal by the first combined stimulation is ignored based on that a magnitude of the first signal has a difference of a predetermined numeric value or more as compared to a magnitude of the second signal.

7. The apparatus of claim 6, wherein
the sensing part is a brain wave measurement part that measures a brain wave of the brain induced corresponding to the stimulation, and
the control part determines whether the first response is derived or not by using a determination of whether a measured brain wave corresponds to an output and a wave form of a entrained brain wave that appears only when entraining the gamma oscillation or not.

8. The apparatus of claim 7, wherein
the control part calculates an average of power spectrum values of respective frequency bands and a standard deviation thereof, and a ratio of each average value according to at least one brain wave combination of gamma (γ)/ alpha (α)/ beta [β)/ delta (δ)/theta (θ) brain waves from the measured brain wave, thus extracting a property of the measured brain wave.

9. The apparatus of claim 8, wherein
when the first response is not derived according to the second combined stimulation, the control part controls the electrical part to transfer, to the brain of the object, a third combined stimulation resulting from additional correction of at least one of the first frequency, the second frequency, and an output, a waveform and a period of a stimulation according to the second frequency.

10. The apparatus of claim 9, wherein
when the first response is not derived according to the first combined stimulation, the control part controls the second combined stimulation according to a 1-1 frequency that is lower than the first frequency of the first combined stimulation to be transferred to the brain of the object,
when the first response is not derived according to the second combined stimulation, the control part controls the third combined stimulation according to a 1-2 frequency that is lower than the 1-1 frequency to be transferred to the brain of the object, and
the 1-1 frequency and the 1-2 frequency are correctable to less than 30 Hz.

11. The apparatus of claim 10, wherein
a local field potential (LFP) of 40 Hz is induced in the prefrontal cortex and the hippocampus of the object through entrainment of the gamma oscillation through stimulation.

12. The apparatus of claim 11, wherein
through entrainment of the gamma oscillation,
a neuronal activity is regulated among the brain areas of the object including the prefrontal cortex and the hippocampus, and
a balance of transmission between GABAergic and glutamatergic neurons that are secreted by excitatory and inhibitory activities of the neuron is induced.

13. The apparatus of claim 12, wherein
through entrainment of the gamma oscillation,
amyloid plaques are reduced in the brain areas of the object including the prefrontal cortex and the hippocampus, and hyperphosphorylation of tau is reduced in the brain areas of the object including the prefrontal cortex and the hippocampus.

14. The apparatus of claim 13, wherein
through entrainment of the gamma oscillation,
losses of neurons and synapses are reduced in the brain areas of the object including the prefrontal cortex and the hippocampus, brain atrophy is reduced in the brain areas of the object including the prefrontal cortex and the hippocampus, atrophy is reduced in the brain areas of the object including the prefrontal cortex and the hippocampus, bulging of a ventricle in the brain is reduced in the brain areas of the object including the prefrontal cortex and the hippocampus, and neuroinflammation is reduced in the brain areas of the object including the prefrontal cortex and the hippocampus.

15. The apparatus of claim 14, wherein
through entrainment of the gamma oscillation,
an immune response of at least a part of microglia is reduce in the brain areas of the object including the prefrontal cortex and the hippocampus, the microglia is deformed morphologically in the brain areas of the object including the prefrontal cortex and the hippocampus, and proteolysis in at least a part of the microglia is increased in the brain areas of the object including prefrontal cortex and hippocampus.

16. A system comprising:
an apparatus; and
a server forming a network with the apparatus, wherein
the apparatus comprises:
a stimulation part that stimulates a brain of an object;
a sensing part that measures a response of the brain to a stimulation; and
a communication part that transmit a measured response of the brain to the server,
the server transmits a first information for the measured response of the brain to a predetermined outside, receives, from the outside, a second information for determination of whether a first response entraining gamma oscillation synchronized in a plurality of brain areas of the object is derived or not based on the measured response of the brain, and transmits the second information to the communication part of the apparatus,
the stimulation part of the apparatus is an electrical stimulation part that transfers a transcranial alternating current stimulation (tACS),
the transcranial alternating current stimulation is a first combined stimulation in which a signal is repeatedly ON/OFF according to a predetermined first frequency and an ON signal according to the first frequency is applied as a stimulation according to a predetermined second frequency,
the outside processes a first signal by the first combine stimulation among signals measured by the sensing part as a noise, and determines whether the first response is derived or not, based on a second signal except the first combined stimulation among the signals measured the sensing part, and
when the first response is not derived according to the first combined stimulation based on the second information, the electrical stimulation part transfers, to the brain of the object, a second combined stimulation resulting from correction of at least one of the first frequency, the second frequency, and an output, a waveform and a period of a stimulation according to the second frequency.

17. A system comprising:
an apparatus; and
a server forming a network with the apparatus, wherein
the apparatus comprises:
a stimulation part that stimulates a brain of an object;
a sensing part that measures a response of the brain to a stimulation;
a control part that determines whether a first response entraining oscillation synchronized in a plurality of brain areas of the object is derived or not, based on a measured brain response; and
a communication part that transmit a measured response of the brain to the server,
the server transmits a first information for the measured response of the brain to a predetermined outside, receives, from the outside, a second information for determination of whether a first response entraining gamma oscillation synchronized in a plurality of brain areas of the object is derived or not based on the measured response of the brain, and transmits the second information to the communication part of the apparatus,
the stimulation part of the apparatus is an electrical stimulation part that transfers a transcranial alternating current stimulation (tACS),
the transcranial alternating current stimulation is a first combined stimulation in which a signal is repeatedly ON/OFF according to a predetermined first frequency and an ON signal according to the first frequency is applied as a stimulation according to a predetermined second frequency,
the control part and the outside of the apparatus processes a first signal by the first combine stimulation among signals measured by the sensing part as a noise, and determines whether the first response is derived or not, based on a second signal except the first combined stimulation among the signals measured the sensing part, and
when the first response is not derived according to the first combined stimulation by using a first determination and a second determination together, the control part controls the electrical stimulation part to transfer, to the brain of the object, a second combined stimulation resulting from correction of at least one of the first frequency, the second frequency, and an output, a waveform and a period of a stimulation according to the second frequency.
